SEQUENCE LISTING (44 sequences)

SEQ ID NO: 1 - DNA, 2151 bp, Arabidopsis thaliana
  gene (1)..(2151): gene for homogentisate-1,2-dioxygenase (HGD)
atggaagagaagaagaaggagcttgaagagttgaagtatcaatcaggttttggtaaccacttctcatcggaagcaatcgccggagctttaccgttagatcagaacagtcctcttctttgtccttacggtctttacgccgaacagatctccggtacttctttcacttctcctcgcaagctcaatcaaagaaggtacatcatcatttcaattgtaagttttggataatttcgttgaattgattgatcttcatcttgttttttttttcagttggttgtaccgggttaaaccatcggttacacatgaaccgttcaagcctcgtgtaccagctcataagaagcttgtgagtgagtttgatgcatcaaatagtcgtacgaatccgactcagcttcggtggagacctgaggatattcctgattcggagattgatttcgttgatgggttatttaccatttgtggagctggaagctcgtttcttcgccatggcttcgctattcacatgtaaaaaactcttctttttattttggtatctttggtgtagatcagtgatacataaagtaatgatcttttgtattcattttgttttgaaggtatgtggctaacacaggaatgaaagactccgcattttgcaacgctgatggtgacttcttgttagttcctcaaacaggaagtaagttagtagtcccaatgccttaccttaccacatctttgggaaataaagtcagtcatgtattgagaatggattcaagatagtcttggatcagttctgatagtttgagtgggtgttttagggctatggattgaaactgagtgtggaaggcttttggtaactcctggtgagattgctgttataccacaaggtttccgtttctccatagatttaccggatgggaagtctcgtggttatgttgctgaaatctatggggctcattttcagcttcctgatcttggaccaataggtactcttgagttcttttagattcagccggaataacatggattctccgcaagaatcttattggtggatgtggacaggtgctaatggtcttgctgcatcaagagattttcttgcaccaacagcatggtttgaggatggattgcggcctgaatacacaattgttcagaagtttggcggtgaactctttactgctaaacaagatttctctccattcaatgtggttgcctggcatggcaattacgtgccttataaggtgagtacattgtttattgagcctaatcttgtaaaacgttaatgcattgtttttctgagaatttcaatttctgtctgcagtatgacctgaagaagttctgtccatacaacactgtgcttttagatcatggagatccatctataaatacaggttggtggtcatctgcgctaaatcgattcttctttttgttttgttatgggttggttacttgttctttattgtaatcacactctttgggtgaattattgtactctcagtccttacagcaccaactgataaacctggtgtggccttgcttgattttgtcatatttcctcctcgatggttggttgctgagcatacttttcgacctccttactatcatcgtaactgcatgagtgaatttatgggcttaatctacggtgcatacgaggtaagctgcttgaagttcctgcttctgcaaatcattagctggcttgtgttatcctcctactgaaatctgtaaactgactccaccattcacaggcgaaagctgatggatttctccctggcggtgcaagtcttcatagctgtatgacacctcatggtccagatactaccacgtacgaggtatcaatccatcttatgcacagcagcaactacacgtttgatttcattttcctccgagatcatgtctaaatctaacccctgaatgtaaaattaagtctgaagcatttttataattgttttgtaggcgacaattgctcgagtaaatgcaatggctccttctaaactcacaggtacgatggctttcatgttcgaatcagcattgatccctagagtctgtcattgggctctggagtctcctttcctggatcacgactactaccagtgttggattggcctcaagtctcatttctcgcgcataagcttggacaagacaaatgttgaatcaacagagaaagaaccaggagcttcggagtaa

SEQ ID NO: 2 - DNA, 1386 bp, Arabidopsis thaliana
  CDS (1)..(1383): cDNA coding for homogentisate-1,2-dioxygenase (HGD)
atggaagagaagaagaaggagcttgaagagttgaagtatcaatcaggttttggtaaccacttctcatcggaagcaatcgccggagctttaccgttagatcagaacagtcctcttctttgtccttacggtctttacgccgaacagatctccggtacttctttcacttctcctcgcaagctcaatcaaagaagttggttgtaccgggttaaaccatcggttacacatgaaccgttcaagcctcgtgtaccagctcataagaagcttgtgagtgagtttgatgcatcaaatagtcgtacgaatccgactcagcttcggtggagacctgaggatattcctgattcggagattgatttcgttgatgggttatttaccatttgtggagctggaagctcgtttcttcgccatggcttcgctattcacatgtatgtggctaacacaggaatgaaagactccgcattttgcaacgctgatggtgacttcttgttagttcctcaaacaggaaggctatggattgaaactgagtgtggaaggcttttggtaactcctggtgagattgctgttataccacaaggtttccgtttctccatagatttaccggatgggaagtctcgtggttatgttgctgaaatctatggggctcattttcagcttcctgatcttggaccaataggtgctaatggtcttgctgcatcaagagattttcttgcaccaacagcatggtttgaggatggattgcggcctgaatacacaattgttcagaagtttggcggtgaactctttactgctaaacaagatttctctccattcaatgtggttgcctggcatggcaattacgtgccttataagtatgacctgaagaagttctgtccatacaacactgtgcttttagatcatggagatccatctataaatacagtccttacagcaccaactgataaacctggtgtggccttgcttgattttgtcatatttcctcctcgatggttggttgctgagcatacttttcgacctccttactatcatcgtaactgcatgagtgaatttatgggcttaatctacggtgcatacgaggcgaaagctgatggatttctccctggcggtgcaagtcttcatagctgtatgacacctcatggtccagatactaccacgtacgaggcgacaattgctcgagtaaatgcaatggctccttctaaactcacaggtacgatggctttcatgttcgaatcagcattgatccctagagtctgtcattgggctctggagtctcctttcctggatcacgactactaccagtgttggattggcctcaagtctcatttctcgcgcataagcttggacaagacaaatgttgaatcaacagagaaagaaccaggagcttcggagtaa

SEQ ID NO: 3 - protein, 461 aa, Arabidopsis thaliana
MEEKKKELEELKYQSGFGNHFSSEAIAGALPLDQNSPLLCPYGLYAEQISGTSFTSPRKLNQRSWLYRVKPSVTHEPFKPRVPAHKKLVSEFDASNSRTNPTQLRWRPEDIPDSEIDFVDGLFTICGAGSSFLRHGFAIHMYVANTGMKDSAFCNADGDFLLVPQTGRLWIETECGRLLVTPGEIAVIPQGFRFSIDLPDGKSRGYVAEIYGAHFQLPDLGPIGANGLAASRDFLAPTAWFEDGLRPEYTIVQKFGGELFTAKQDFSPFNVVAWHGNYVPYKYDLKKFCPYNTVLLDHGDPSINTVLTAPTDKPGVALLDFVIFPPRWLVAEHTFRPPYYHRNCMSEFMGLIYGAYEAKADGFLPGGASLHSCMTPHGPDTTTYEATIARVNAMAPSKLTGTMAFMFESALIPRVCHWALESPFLDHDYYQCWIGLKSHFSRISLDKTNVESTEKEPGASE

SEQ ID NO: 4 - DNA, 1227 bp, Arabidopsis thaliana
  CDS (1)..(1224): cDNA coding for fumarylacetoacetate hydrolase (FAAH)
atggcgttgctgaagtctttcatcgatgttggctcagactcgcacttccctatccagaatctcccttatggtgtcttcaaaccggaatcgaactcaactcctcgtcctgccgtcgctatcggcgatttggttctggacctctccgctatctctgaagctgggcttttcgatggtctgatccttaaggacgcagattgctttcttcagcctaatttgaataagttcttggccatgggacggcctgcgtggaaggaagcgcgttctacgctgcaaagaatcttgtcatttttgttatttggcttcaaggttttggttttggtatgttttcatgcagctaatgaacctatcttgcgagacaatgatgttttgaggagaaaatcattccatcagatgagtaaagtggaaatgattgttcctatggtgattggggactatacagacttctttgcatctatgcatcacgcgaagaactgcggacttatgttccgtgggcctgagaatgcgataaacccaaattggtttcgtcttcccattgcatatcatggacgggcatcatctattgtcatctctgggactgacattattcgaccaagaggtcagggccatccacaaggaaactctgaaccatattttggaccttcgaagaaacttgattttgagcttgagatggctgctgtggttggtccaggaaatgaattgggaaagcctattgacgtgaataatgcagccgatcatatatttggtctattactgatgaatgactggagtgctagggatattcaggcgtgggagtatgtacctcttggtcctttcctggggaagagttttgggactactatatccccttggattgttaccttggatgcgcttgagccttttggttgtcaagctcccaagcaggatccacctccattgccatatttggctgagaaagagtctgtaaattacgatatctccttggagctagcacaccataccgttaacggttgcaatttgaggcctggtgatctccttggcacaggaaccataagcggaccggagccagattcatatgggtgcctacttgagttgacatggaatggacagaaacctctatcactcaatggaacaactcagacgtttctcgaagacggagaccaagtcaccttctcaggtgtatgcaagggagatggttacaatgttgggtttggaacatgcacagggaaaattgttccttcaccgccttga

SEQ ID NO: 5 - protein, 408 aa, Arabidopsis thaliana
MALLKSFIDVGSDSHFPIQNLPYGVFKPESNSTPRPAVAIGDLVLDLSAISEAGLFDGLILKDADCFLQPNLNKFLAMGRPAWKEARSTLQRILSFLLFGFKVLVLVCFHAANEPILRDNDVLRRKSFHQMSKVEMIVPMVIGDYTDFFASMHHAKNCGLMFRGPENAINPNWFRLPIAYHGRASSIVISGTDIIRPRGQGHPQGNSEPYFGPSKKLDFELEMAAVVGPGNELGKPIDVNNAADHIFGLLLMNDWSARDIQAWEYVPLGPFLGKSFGTTISPWIVTLDALEPFGCQAPKQDPPPLPYLAEKESVNYDISLELAHHTVNGCNLRPGDLLGTGTISGPEPDSYGCLLELTWNGQKPLSLNGTTQTFLEDGDQVTFSGVCKGDGYNVGFGTCTGKIVPSPP

SEQ ID NO: 6 - DNA, 1721 bp, Arabidopsis thaliana
  gene (9)..(1713): gene for maleylacetoacetate isomerase (MAAI)
atgtcgacatgtcttatgttaccgatttttatcaggcgaagttgaagctctactcttactggagaagctcatgtgctcatcgcgtccgtatcgccctcactttaaaaggtaccagccaatgattttattcttttcttgtgagcaattctttgatctgaatttggttcttgttcgattttcattagggcttgattatgaatatataccggttaatttgctcaaaggggatcaatccgattcaggtgcgtagtttctaggttatattgaactttatttgaagtaacattgtaaagataagaatggtaagtaactgagatttcttatgttagacttagaagtttattcgttttggttctctagatttcaagaagatcaatccaatgggcactgtaccagcgcttgttgatggtgatgttgtgattaatgactctttcgcaataataatggtcagtagtaacacatccatttagtttgtttggttttgttgatgaaaaggaacattcgtttattcgtcttgttgtttttcaaatggacagtacctggatgataagtatccggagccaccgctgttaccaagtgactaccataaacgggcggtaaattaccaggtatcttcgatcctttgtcttcagatgatgatgtgttgccatcatctgcaaaaccatgtagttaagtccaaatgtagtgaacattatcagctttagattgcgagtgtgatcgttgttcttattttgtatatttcaggcgacgagtattgtcatgtctggtatacagcctcatcaaaatatggctctttttgtgagaagatgagattaatgtaatggattctactaatggaggttctataacaaagcaaacatagttacattttgtcattttttttaacagaggtatctcgaggacaagataaatgctgaggagaaaactgcttggattactaatgctatcacaaaaggattcacaggtatgatatctctaatctacctatacgtaatcaagaaccaagacatatgttcaaaatgtgattttgttgatattgtggttgtacaggtttataacgacctgtctgataatgtctcatatgtccttcagctctcgagaaactgttggtgagttgcgctggaaaatacgcgactggtgatgaagtttacttggtatgtctctaaatctccctggataatctctatggtactactctcttctttattacaatgaagcattgttttgcaggctgatcttttcctagcaccacagatccacgcagcattcaacagattccatattaacatggtacttttcctcagctaatctcttctcctggtacctagatattgcattgtatatccccccaaattccatggaatccttgatcagagttttaaggtagcatgaaccaaatgttatctctgtctcacactttcacattcacagagtaacatagacgtaatactcagtttcataactttttttcctcgcatcacttggttttcatctctacaattttgttgtataggaaccattcccgactcttgcaaggttttacgagtcatacaacgaactgcctgcatttcaaaatgcagtcccggagaagcaaccagatactccttccaccatctgattctgtgaaccgtaagcttctctcagtctcagctcaataaaatctcttaggaaacaacaacaacaccttgaacttaaatgtatcatatgaaccagggatccat

SEQ ID NO: 7 - DNA, 1238 bp, Escherichia coli
  gene (7)..(1232): tyrA gene coding for bifunctional chorismate mutase / prephenate dehydrogenase
cccgggtggcttaagaggtttattatggttgctgaattgaccgcattacgcgatcaaattgatgaagtcgataaagcgctgctgaatttattagcgaagcgtctggaactggttgctgaagtgggcgaggtgaaaagccgctttggactgcctatttatgttccggagcgcgaggcatctatgttggcctcgcgtcgtgcagaggcggaagctctgggtgtaccgccagatctgattgaggatgttttgcgtcgggtgatgcgtgaatcttactccagtgaaaacgacaaaggatttaaaacactttgtccgtcactgcgtccggtggttatcgtcggcggtggcggtcagatgggacgcctgttcgagaagatgctgaccctctcgggttatcaggtgcggattctggagcaacatgactgggatcgagcggctgatattgttgccgatgccggaatggtgattgttagtgtgccaatccacgttactgagcaagttattggcaaattaccgcctttaccgaaagattgtattctggtcgatctggcatcagtgaaaaatgggccattacaggccatgctggtggcgcatgatggtccggtgctggggctacacccgatgttcggtccggacagcggtagcctggcaaagcaagttgtggtctggtgtgatggacgtaaaccggaagcataccaatggtttctggagcaaattcaggtctggggcgctcggctgcatcgtattagcgccgtcgagcacgatcagaatatggcgtttattcaggcactgcgccactttgctacttttgcttacgggctgcacctggcagaagaaaatgttcagcttgagcaacttctggcgctctcttcgccgatttaccgccttgagctggcgatggtcgggcgactgtttgctcaggatccgcagctttatgccgacatcattatgtcgtcagagcgtaatctggcgttaatcaaacgttactataagcgtttcggcgaggcgattgagttgctggagcagggcgataagcaggcgtttattgacagtttccgcaaggtggagcactggttcggcgattacgcacagcgttttcagagtgaaagccgcgtgttattgcgtcaggcgaatgacaatcgccagtaataatccagtgccggatgattcacatcatccggcaccttttcatcaggttggatcaacaggcactacgttctcacttgggtaacagcgtcgac

SEQ ID NO: 8 - protein, 373 aa, Escherichia coli
MVAELTALRDQIDEVDKALLNLLAKRLELVAEVGEVKSRFGLPIYVPEREASMLASRRAEAEALGVPPDLIEDVLRRVMRESYSSENDKGFKTLCPSLRPVVIVGGGGQMGRLFEKMLTLSGYQVRILEQHDWDRAADIVADAGMVIVSVPIHVTEQVIGKLPPLPKDCILVDLASVKNGPLQAMLVAHDGPVLGLHPMFGPDSGSLAKQVVVWCDGRKPEAYQWFLEQIQVWGARLHRISAVEHDQNMAFIQALRHFATFAYGLHLAEENVQLEQLLALSSPIYRLELAMVGRLFAQDPQLYADIIMSSERNLALIKRYYKRFGEAIELLEQGDKQAFIDSFRKVEHWFGDYAQRFQSESRVLLRQANDNRQ

SEQ ID NO: 9 - DNA, 2953 bp, Arabidopsis thaliana
  gene (1)..(2953): gene for fumarylacetoacetate hydrolase (FAAH)
atggcgttgctgaagtctttcatcgatgttggctcagactcgcacttccctatccagaatctcccttatggtgtcttcaaaccggaatcgaactcaactcctcgtcctgccgtcgctatcggcgatttggttctggacctctccgctatctctgaagctgggcttttcgatggtctgatccttaaggacgcagattgctttcttcaggttcgtttttccgattcctataaactcggattactatgtagtagtaccctgggaatgtttccgtaaatgatttcgaatttgctatttgaacctgatctctgaagtttgctccatggtttattggatagatcaatcccgtttagctcgaaaaaaatccattgttctactcaattgctcgttgcttcgattcattatctgttacagtttgagttttctgttcacgattttgaacttttgcaactatgattattgctttatgatctgacggtatagtgtattgcttacacttagtgatgaggaaaatgaggttgtgtttattttctggtgtgtttcttttgatgttaatattgtttagtttctgtgctctgtttgcagcctaatttgaataagttcttggccatgggacggcctgcgtggaaggaagcgcgttctacgctgcaaagaatcttgtcatgtatgctctgtttgatcctattgatttatttggatttttatggagttttgttatttggcttcaaggttttggttttggtatgttttcatgcagctaatgaacctatcttgcgagacaatgatgttttgaggagaaaatcattccatcagatggttagtagtgtgaaattgttttttgcttaaactagggaaattgtttgtatatctgttacttacgtttattgctgtttgatgcaaatttgcagagtaaagtggaaatgattgttcctatggtgattggggactatacagacttctttgcatctatgcatcacgcgaagaactgcggacttatgttccgtgggcctgagaatgcgataaacccaaattggtgcgtttatgttacttttgagctgagagtttcttcatgaaatggtcaagtcgaaaggatgactctgtattaacatgacattaccatatttttcaggtttcgtcttcccattgcatatcatggacgggcatcatctattgtcatctctgggactgacattattcgaccaaggttaggaaattgtgtattattatctggtttttggtgggctgagaatggttgttaagaataattcacatgtcatatttgaagtcatgcatcatgcaaggttttatgctttgacaagaaatatagttttttataagatattattacattgaaaccaatattggcggatggtaaaatttcatgcagacaaattaataatgaaatgctaattccagttttatctttgcttgttttgctttcttccagaggtcagggccatccacaaggaaactctgaaccatattttggaccttcgaagaaacttgattttgagcttgagatggtaagcatctgatgcctcagttatgtggatttgttttacaatgattcggttgatgctttttggtgctagttaagaataacggcattgacaaacctctcttttatcacatgatattcaggctgctgtggttggtccaggaaatgaattgggaaagcctattgacgtgaataatgcagccgatcatatatttggtctattactgatgaatgactggagtggtactcacttaactatagttttcgttgagtcatctttaacctgaccgggcatgaccggtttttttaaatgtttgttgttatagctagggatattcaggcgtgggagtatgtacctcttggtcctttcctggggaagagttttggtgagatatttggcttcaatactttgatttcatttcctctagttgaagtatatgggcaaagaacttcggtgaatgttgtcttgttgtgttgtagggactactatatccccttggattgttaccttggatgcgcttgagccttttggttgtcaagctcccaagcaggttggtacttaggcatcacattctttttgtgtcacgcaatcactgattctctcatgatctaacttgttcttggggcaggatccacctccattgccatatttggctgagaaagagtctgtaaattacgatatctccttggaggtagcattcgatattggagtttcactttttggctttttgctatcaactataacagcttatggtggactgaactgaaataaacatcatgtttttacctcttataggttcaacttaaaccttctggcagagatgattcttgtgtaataacaaagagcaacttccaaaacttgtgagttcctctataatctcctacccaattcctccatataattaaacagtttggttcaaactcttttaaacttattgtgacagatattggaccataacgcagcagctagcacaccataccgttaacggttgcaatttgaggcctggtgatctccttggcacaggaaccataagcggaccggtaaactcttttcgaaccagttctctcgtctactatatcacgtgatgactacacaataactcgcaaaatctttgtttcttggttctaaacgcaggagccagattcatatgggtgcctacttgagttgacatggaatggacagaaacctctatcactcaatggaacaactcagacgtttctcgaagacggagaccaagtcaccttctcaggtgtatgcaaggtatcagctgattaacacggtttctgctttagtttaatttgctttataccccaacaactccaaatgaatttcgttgcatgacatttcggttaacgcttattaatcaaattacgtctatgattaaaccgttgtagggagatggttacaatgttgggtttggaacatgcacagggaaaattgttccttcaccgccttga

SEQ ID NO: 10 - DNA, 1534 bp, Arabidopsis thaliana
  CDS (28)..(1227): cDNA coding for hydroxyphenylpyruvate dioxygenase
cgagttttagcagagttggtgaaatcaatgggccaccaaaacgccgccgtttcagagaatcaaaaccatgatgacggcgctgcgtcgtcgccgggattcaagctcgtcggattttccaagttcgtaagaaagaatccaaagtctgataaattcaaggttaagcgcttccatcacatcgagttctggtgcggcgacgcaaccaacgtcgctcgtcgcttctcctggggtctggggatgagattctccgccaaatccgatctttccaccggaaacatggttcacgcctcttacctactcacctccggtgacctccgattccttttcactgctccttactctccgtctctctccgccggagagattaaaccgacaaccacagcttctatcccaagtttcgatcacggctcttgtcgttccttcttctcttcacatggtctcggtgttagagccgttgcgattgaagtagaagacgcagagtcagctttctccatcagtgtagctaatggcgctattccttcgtcgcctcctatcgtcctcaatgaagcagttacgatcgctgaggttaaactatacggcgatgttgttctccgatatgttagttacaaagcagaagataccgaaaaatccgaattcttgccagggttcgagcgtgtagaggatgcgtcgtcgttcccattggattatggtatccggcggcttgaccacgccgtgggaaacgttcctgagcttggtccggctttaacttatgtagcggggttcactggttttcaccaattcgcagagttcacagcagacgacgttggaaccgccgagagcggtttaaattcagcggtcctggctagcaatgatgaaatggttcttctaccgattaacgagccagtgcacggaacaaagaggaagagtcagattcagacgtatttggaacataacgaaggcgcagggctacaacatctggctctgatgagtgaagacatattcaggaccctgagagagatgaggaagaggagcagtattggaggattcgacttcatgccttctcctccgcctacttactaccagaatctcaagaaacgggtcggcgacgtgctcagcgatgatcagatcaaggagtgtgaggaattagggattcttgtagacagagatgatcaagggacgttgcttcaaatcttcacaaaaccactaggtgacaggtacagttcatttaatcaaacacatgttacagttccctaacaatccatttgatgataaacatgttacagtttactaagcaatctcttgtttatgattgtgttaataggccgacgatatttatagagataatccagagagtaggatgcatgatgaaagatgaggaagggaaggcttaccagagtggaggatgtggtggtctctgagctcttcaagtccattgaagaatacgaaaagactcttgaagccaaacagttagtgggatgaacaagaagaagaaccaactaaaggattgtgtaattaatgtaaaactgttttatcttatcaaaacaatgttatacaacat

SEQ ID NO: 11 - protein, 400 aa, Arabidopsis thaliana
MGHQNAAVSENQNHDDGAASSPGFKLVGFSKFVRKNPKSDKFKVKRFHHIEFWCGDATNVARRFSWGLGMRFSAKSDLSTGNMVHASYLLTSGDLRFLFTAPYSPSLSAGEIKPTTTASIPSFDHGSCRSFFSSHGLGVRAVAIEVEDAESAFSISVANGAIPSSPPIVLNEAVTIAEVKLYGDVVLRYVSYKAEDTEKSEFLPGFERVEDASSFPLDYGIRRLDHAVGNVPELGPALTYVAGFTGFHQFAEFTADDVGTAESGLNSAVLASNDEMVLLPINEPVHGTKRKSQIQTYLEHNEGAGLQHLALMSEDIFRTLREMRKRSSIGGFDFMPSPPPTYYQNLKKRVGDVLSDDQIKECEELGILVDRDDQGTLLQIFTKPLGDRYSSFNQTHVTVP

SEQ ID NO: 12 - DNA, 575 bp, Brassica napus
  misc_feature (1)..(6): restriction site
gtcgacgggccgatgggggcgaagggtcttgctgcaccaagagattttcttgcaccaacggcatggtttgaggaagggctacggcctgactacactattgttcagaagtttggcggtgaactctttactgctaaacaagatttctctccgttcaatgtggttgcctggcatggcaattacgtgccttataagtatgacctgcacaagttctgtccatacaacactgtccttgtagaccatggagatccatctgtaaatacagttctgacagcaccaacggataaacctggtgtggccttgcttgattttgtcatattccctcctcgttggttggttgctgagcatacctttcgacctccttactaccatcgtaactgcatgagtgaatttatgggcctaatctatggtgcttacgaggccaaagctgatggatttctacctggtggcgcaagtcttcacagttgtatgacacctcatggtccagatacaaccacatacgaggcgacgattgctcgtgtaaatgcaatggctccttataagctcacaggcaccatggccttcatgtttgaggtacc

SEQ ID NO: 13 - DNA, 932 bp, Synechocystis PCC6803
  CDS (4)..(927): cDNA coding for homogentisate phytyltransferase
gccatggcaactatccaagctttttggcgcttctcccgcccccataccatcattggtacaactctgagcgtctgggctgtgtatctgttaactattctcggggatggaaactcagttaactcccctgcttccctggatttagtgttcggcgcttggctggcctgcctgttgggtaatgtgtacattgtcggcctcaaccaattgtgggatgtggacattgaccgcatcaataagccgaatttgcccctagctaacggagatttttctatcgcccagggccgttggattgtgggactttgtggcgttgcttccttggcgatcgcctggggattagggctatggctggggctaacggtgggcattagtttgattattggcacggcctattcggtgccgccagtgaggttaaagcgcttttccctgctggcggccctgtgtattctgacggtgcggggaattgtggttaacttgggcttatttttattttttagaattggtttaggttatccccccactttaataacccccatctgggttttgactttatttatcttagttttcaccgtggcgatcgccatttttaaagatgtgccagatatggaaggcgatcggcaatttaagattcaaactttaactttgcaaatcggcaaacaaaacgtttttcggggaaccttaattttactcactggttgttatttagccatggcaatctggggcttatgggcggctatgcctttaaatactgctttcttgattgtttcccatttgtgcttattagccttactctggtggcggagtcgagatgtacacttagaaagcaaaaccgaaattgctagtttttatcagtttatttggaagctatttttcttagagtacttgctgtatcccttggctctgtggttacctaatttttctaatactattttttaggg

SEQ ID NO: 14 - protein, 308 aa, Synechocystis PCC6803
MATIQAFWRFSRPHTIIGTTLSVWAVYLLTILGDGNSVNSPASLDLVFGAWLACLLGNVYIVGLNQLWDVDIDRINKPNLPLANGDFSIAQGRWIVGLCGVASLAIAWGLGLWLGLTVGISLIIGTAYSVPPVRLKRFSLLAALCILTVRGIVVNLGLFLFFRIGLGYPPTLITPIWVLTLFILVFTVAIAIFKDVPDMEGDRQFKIQTLTLQIGKQNVFRGTLILLTGCYLAMAIWGLWAAMPLNTAFLIVSHLCLLALLWWRSRDVHLESKTEIASFYQFIWKLFFLEYLLYPLALWLPNFSNTIF

SEQ ID NO: 15 - DNA, 1159 bp, Artificial sequence
  CDS (8)..(1150)
  misc_feature (1)..(6): restriction site
gtcgactatgactcaaactactcatcatactccagatactgctagacaagctgatccttttccagttaagggaatggatgctgttgttttcgctgttggaaacgctaagcaagctgctcattactactctactgctttcggaatgcaacttgttgcttactctggaccagaaaacggatctagagaaactgcttcttacgttcttactaacggatctgctagattcgttcttacttctgttattaagccagctaccccatggggacatttccttgctgatcacgttgctgaacacggagatggagttgttgatcttgctattgaagttccagatgctagagctgctcatgcttacgctattgaacatggagctagatctgttgctgaaccatacgaacttaaggatgaacatggaactgttgttcttgctgctattgctacttacggaaagactagacatactcttgttgatagaactggatacgatggaccataccttccaggatacgttgctgctgctccaattgttgaaccaccagctcatagaaccttccaagctattgaccattgtgttggtaacgttgaactcggaagaatgaacgaatgggttggattctacaacaaggttatgggattcactaacatgaaggaattcgttggagatgatattgctactgagtactctgctcttatgtctaaggttgttgctgatggaactcttaaggttaaattcccaattaatgaaccagctcttgctaagaagaagtctcagattgatgaataccttgagttctacggaggagctggagttcaacatattgctcttaacactggagatatcgtggaaactgttagaactatgagagctgcaggagttcaattccttgatactccagattcttactacgatactcttggtgaatgggttggagatactagagttccagttgatactcttagagaacttaagattcttgctgatagagatgaagatggataccttcttcaaatcttcactaagccagttcaagatagaccaactgtgttcttcgaaatcattgaaagacatggatctatgggattcggaaagggtaacttcaaggctcttttcgaagctattgaaagagaacaagagaagagaggaaacctttaggtcgac

SEQ ID NO: 16 - protein, 381 aa, Artificial sequence
  Description of the artificial sequence codon usage optimized cDNA coding for hydroxyphenylpyruvate dioxygenase from Streptomyces avermitilis
MTQTTHHTPDTARQADPFPVKGMDAVVFAVGNAKQAAHYYSTAFGMQLVAYSGPENGSRETASYVLTNGSARFVLTSVIKPATPWGHFLADHVAEHGDGVVDLAIEVPDARAAHAYAIEHGARSVAEPYELKDEHGTVVLAAIATYGKTRHTLVDRTGYDGPYLPGYVAAAPIVEPPAHRTFQAIDHCVGNVELGRMNEWVGFYNKVMGFTNMKEFVGDDIATEYSALMSKVVADGTLKVKFPINEPALAKKKSQIDEYLEFYGGAGVQHIALNTGDIVETVRTMRAAGVQFLDTPDSYYDTLGEWVGDTRVPVDTLRELKILADRDEDGYLLQIFTKPVQDRPTVFFEIIERHGSMGFGKGNFKALFEAIEREQEKRGNL

SEQ ID NO: 17 - DNA, 815 bp, Arabidopsis thaliana
  CDS (37)..(705): cDNA coding for maleylcetoacetate isomerase
gtaatctccgaagaagaacaaattccttgctgaatcatgtcttatgttaccgatttttatcaggcgaagttgaagctctactcttactggagaagctcatgtgctcatcgcgtccgtatcgccctcactttaaaagggcttgattatgaatatataccggttaatttgctcaaaggggatcaatccgattcagatttcaagaagatcaatccaatgggcactgtaccagcgcttgttgatggtgatgttgtgattaatgactctttcgcaataataatgtacctggatgataagtatccggagccaccgctgttaccaagtgactaccataaacgggcggtaaattaccaggcgacgagtattgtcatgtctggtatacagcctcatcaaaatatggctctttttaggtatctcgaggacaagataaatgctgaggagaaaactgcttggattactaatgctatcacaaaaggattcacagctctcgagaaactgttggtgagttgcgctggaaaatacgcgactggtgatgaagtttacttggctgatcttttcctagcaccacagatccacgcagcattcaacagattccatattaacatggaaccattcccgactcttgcaaggttttacgagtcatacaacgaactgcctgcatttcaaaatgcagtcccggagaagcaaccagatactccttccaccatctgattctgtgaaccgtaagcttctctcagtctcagctcaataaaatctcttaggaaacaacaacaacaccttgaacttaaatgtatcatatgaaccagtttacaaataat

SEQ ID NO: 18 - protein, 223 aa, Arabidopsis thaliana
MSYVTDFYQAKLKLYSYWRSSCAHRVRIALTLKGLDYEYIPVNLLKGDQSDSDFKKINPMGTVPALVDGDVVINDSFAIIMYLDDKYPEPPLLPSDYHKRAVNYQATSIVMSGIQPHQNMALFRYLEDKINAEEKTAWITNAITKGFTALEKLLVSCAGKYATGDEVYLADLFLAPQIHAAFNRFHINMEPFPTLARFYESYNELPAFQNAVPEKQPDTPSTI

SEQ ID NO: 19 - DNA, 1350 bp, Arabidopsis thaliana
  CDS (63)..(1106): coding for gamma-tocopherol methyltransferase
ccacgcgtccgcaaataatccctgacttcgtcacgtttctttgtatctccaacgtccaataaatgaaagcaactctagcagcaccctcttctctcacaagcctcccttatcgaaccaactcttctttcggctcaaagtcatcgcttctctttcggtctccatcctcctcctcctcagtctctatgacgacaacgcgtggaaacgtggctgtggcggctgctgctacatccactgaggcgctaagaaaaggaatagcggagttctacaatgaaacttcgggtttgtgggaagagatttggggagatcatatgcatcatggcttttatgaccctgattcttctgttcaactttctgattctggtcacaaggaagctcagatccgtatgattgaagagtctctccgtttcgccggtgttactgatgaagaggaggagaaaaagataaagaaagtagtggatgttgggtgtgggattggaggaagctcaagatatcttgcctctaaatttggagctgaatgcattggcattactctcagccctgttcaggccaagagagccaatgatctcgcggctgctcaatcactctctcataaggcttccttccaagttgcggatgcgttggatcagccattcgaagatggaaaattcgatctagtgtggtcgatggagagtggtgagcatatgcctgacaaggccaagtttgtaaaagagttggtacgtgtggcggctccaggaggtaggataataatagtgacatggtgccatagaaatctatctgcgggggaggaagctttgcagccgtgggagcaaaacatcttggacaaaatctgtaagacgttctatctcccggcttggtgctccaccgatgattatgtcaacttgcttcaatcccattctctccaggatattaagtgtgcggattggtcagagaacgtagctcctttctggcctgcggttatacggactgcattaacatggaagggccttgtgtctctgcttcgtagtggtatgaaaagtattaaaggagcattgacaatgccattgatgattgaaggttacaagaaaggtgtcattaagtttggtatcatcacttgccagaagccactctaagtctaaagctatactaggagattcaataagactataagagtagtgtctcatgtgaaagcatgaaattccttaaaaacgtcaatgttaagcctatgcttcgttatttgttttagataagtatcatttcactcttgtctaaggtagtttctataaacaataaataccatgaattagctcatgttatctggtaaattctcggaagtgattgtcatggattaactcaaaaaaaaaaaaaaaaaaa

SEQ ID NO: 20 - protein, 348 aa, Arabidopsis thaliana
MKATLAAPSSLTSLPYRTNSSFGSKSSLLFRSPSSSSSVSMTTTRGNVAVAAAATSTEALRKGIAEFYNETSGLWEEIWGDHMHHGFYDPDSSVQLSDSGHKEAQIRMIEESLRFAGVTDEEEEKKIKKVVDVGCGIGGSSRYLASKFGAECIGITLSPVQAKRANDLAAAQSLSHKASFQVADALDQPFEDGKFDLVWSMESGEHMPDKAKFVKELVRVAAPGGRIIIVTWCHRNLSAGEEALQPWEQNILDKICKTFYLPAWCSTDDYVNLLQSHSLQDIKCADWSENVAPFWPAVIRTALTWKGLVSLLRSGMKSIKGALTMPLMIEGYKKGVIKFGIITCQKPL

SEQ ID NO: 21 - DNA, 957 bp, Synechocystis PCC6803
  CDS (1)..(954): cDNA coding for 2-methyl-6-phytylhydrochinone methyltransferase
atgcccgagtatttgcttctgcccgctggcctaatttccctctccctggcgatcgccgctggactgtatctcctaactgcccggggctatcagtcatcggattccgtggccaacgcctacgaccaatggacagaggacggcattttggaatattactggggcgaccatatccacctcggccattatggcgatccgccagtggccaaggatttcatccaatcgaaaattgattttgtccatgccatggcccagtggggcggattagatacacttccccccggcacaacggtattggatgtgggttgcggcattggcggtagcagtcgcattctcgccaaagattatggttttaacgttaccggcatcaccattagtccccaacaggtgaaacgggcgacggaattaactcctcccgatgtgacggccaagtttgcggtggacgatgctatggctttgtcttttcctgacggtagtttcgacgtagtttggtcggtggaagcagggccccacatgcctgacaaagctgtgtttgccaaggaattactgcgggtcgtgaaaccagggggcattctggtggtggcggattggaatcaacgggacgatcgccaagtgcccctcaacttctgggaaaaaccagtgatgcgacaactgttggatcaatggtcccaccctgcctttgccagcattgaaggttttgcggaaaatttggaagccacgggtttggtggagggccaggtgactactgctgattggactgtaccgaccctccccgcttggttggataccatttggcagggcattatccggccccagggctggttacaatacggcattcgtgggtttatcaaatccgtgcgggaagtaccgactattttattgatgcgccttgcctttggggtaggactttgtcgcttcggtatgttcaaagcagtgcgaaaaaacgccactcaagcttaa

SEQ ID NO: 22 - protein, 318 aa, Synechocystis PCC6803
MPEYLLLPAGLISLSLAIAAGLYLLTARGYQSSDSVANAYDQWTEDGILEYYWGDHIHLGHYGDPPVAKDFIQSKIDFVHAMAQWGGLDTLPPGTTVLDVGCGIGGSSRILAKDYGFNVTGITISPQQVKRATELTPPDVTAKFAVDDAMALSFPDGSFDVVWSVEAGPHMPDKAVFAKELLRVVKPGGILVVADWNQRDDRQVPLNFWEKPVMRQLLDQWSHPAFASIEGFAENLEATGLVEGQVTTADWTVPTLPAWLDTIWQGIIRPQGWLQYGIRGFIKSVREVPTILLMRLAFGVGLCRFGMFKAVRKNATQA

SEQ ID NO: 23 - DNA, 1395 bp, Nicotiana tabacum
  CDS (1)..(1392): cDNA coding for geranylgeranylpyrophosphate oxidoreductase
atggcttccattgctctcaaaactttcaccggcctccgtcaatcctcgccggaaaacaattccattactctttctaaatccctccccttcacccaaacccaccgtaggctccgaatcaatgcttccaaatccagcccaagagtcaacggccgcaatcttcgtgttgcggtggtgggcggtggtcctgctggtggcgccgccgctgaaacactcgccaagggaggaattgaaaccttcttaatcgaacgcaaaatggacaactgcaaaccctgcggtggggccatcccactttgcatggtgggagaatttgacctccctttggatatcattgaccggaaagttacaaagatgaagatgatttccccatccaacgttgctgttgatattggtcagactttaaagcctcacgagtacatcggtatggtgcgccgcgaagtactcgatgcttacctccgtgaccgcgctgctgaagccggagcctctgttctcaacggcttgttcctcaaaatggacatgcccaaagctcccaacgcaccttacgtccttcactacacagcttacgactccaaaactaatggcgcgggggagaagcgtaccctggaagttgacgccgttatcggcgctgacggtgcaaattcccgtgtcgcaaaatccataaacgccggtgactacgagtacgctattgcattccaagaaaggattaaaatttccgatgataaaatgaagtattacgagaatttagctgaaatgtacgtgggtgatgacgtgtcccctgatttttacgggtgggttttccccaaatgtgaccacgttgccgttggcactggcacagtcacccacaaagctgacatcaaaaaattccagctagctacaagattgagagctgattccaaaatcaccggcggaaaaattatccgggtcgaggcccacccgattccagaacacccaagacccagaagattacaagacagagttgcattggttggtgatgcggcagggtacgtgaccaaatgttcgggcgaagggatttacttcgcggcaaagagtggacgtatgtgtgctgaagcaattgttgaagggtcagaaatgggaaaaagaatggtggacgagagtgatttgaggaagtatttggagaaatgggacaagacttattggccaacgtacaaggtgcttgatatattgcagaaggtattttacaggtcgaatccggcgagggaagcatttgttgaaatgtgcgcagatgagtatgtgcagaagatgacatttgacagctatttgtacaagaaagtagcaccaggaaacccaattgaagacttgaagcttgctgtgaataccattggaagtttggtgagagctaatgcactaagaagggaaatggacaagctcagtgtataa

SEQ ID NO: 24 - protein, 464 aa, Nicotiana tabacum
MASIALKTFTGLRQSSPENNSITLSKSLPFTQTHRRLRINASKSSPRVNGRNLRVAVVGGGPAGGAAAETLAKGGIETFLIERKMDNCKPCGGAIPLCMVGEFDLPLDIIDRKVTKMKMISPSNVAVDIGQTLKPHEYIGMVRREVLDAYLRDRAAEAGASVLNGLFLKMDMPKAPNAPYVLHYTAYDSKTNGAGEKRTLEVDAVIGADGANSRVAKSINAGDYEYAIAFQERIKISDDKMKYYENLAEMYVGDDVSPDFYGWVFPKCDHVAVGTGTVTHKADIKKFQLATRLRADSKITGGKIIRVEAHPIPEHPRPRRLQDRVALVGDAAGYVTKCSGEGIYFAAKSGRMCAEAIVEGSEMGKRMVDESDLRKYLEKWDKTYWPTYKVLDILQKVFYRSNPAREAFVEMCADEYVQKMTFDSYLYKKVAPGNPIEDLKLAVNTIGSLVRANALRREMDKLSV

SEQ ID NO: 25 - DNA, 26 bp, oligonucleotide
  misc_feature (9): A, T, G or C
gtcgacggnccnatnggngcnaangg

SEQ ID NO: 26 - DNA, 24 bp, oligonucleotide
aagcttccgatctagtaacataga

SEQ ID NO: 27 - DNA, 32 bp, oligonucleotide
attctagacatggagtcaaagattcaaataga

SEQ ID NO: 28 - DNA, 32 bp, oligonucleotide
attctagaggacaatcagtaaattgaacggag

SEQ ID NO: 29 - DNA, 26 bp, oligonucleotide
atgtcgacatgtcttatgttaccgat

SEQ ID NO: 30 - DNA, 25 bp, oligonucleotide
atggatccctggttcatatgataca

SEQ ID NO: 31 - DNA, 26 bp, oligonucleotide
atgtcgacggaaactctgaaccatat

SEQ ID NO: 32 - DNA, 25 bp, oligonucleotide
atggtaccgaatgtgatgcctaagt

SEQ ID NO: 33 - DNA, 29 bp, oligonucleotide
  misc_feature (18): A, T, G or C
ggtacctcraacatraangccatngtncc

SEQ ID NO: 34 - DNA, 25 bp, oligonucleotide
gaattcgatctgtcgtctcaaactc

SEQ ID NO: 35 - DNA, 26 bp, oligonucleotide
ggtaccgtgatagtaaacaactaatg

SEQ ID NO: 36 - DNA, 34 bp, oligonucleotide
atggtaccttttttgcataaacttatcttcatag

SEQ ID NO: 37 - DNA, 43 bp, oligonucleotide
atgtcgacccgggatccagggccctgatgggtcccattttccc

SEQ ID NO: 38 - DNA, 25 bp, oligonucleotide
gtcgacgaatttccccgaatcgttc

SEQ ID NO: 39 - DNA, 24 bp, oligonucleotide
aagcttccgatctagtaacataga

SEQ ID NO: 40 - DNA, 25 bp, oligonucleotide
aagcttgatctgtcgtctcaaactc

SEQ ID NO: 41 - DNA, 1721 bp, Arabidopsis thaliana; identical to SEQ ID NO: 6
  misc_feature (1)..(8): restriction site linker

SEQ ID NO: 42 - DNA, 622 bp, Arabidopsis thaliana
  misc_feature (1)..(8): restriction site linker
atgtcgacggaaactctgaaccatattttggaccttcgaagaaacttgattttgagcttgagatggtaagcatctgatgcctcagttatgtggatttgttttacaatgattcggttgatgctttttggtgctagttaagaataacggcattgacaaacctctcttttatcacatgatattcaggctgctgtggttggtccaggaaatgaattgggaaagcctattgacgtgaataatgcagccgatcatatatttggtctattactgatgaatgactggagtggtactcacttaactatagttttcgttgagtcatctttaacctgaccgggcatgaccggtttttttaaatgtttgttgttatagctagggatattcaggcgtgggagtatgtacctcttggtcctttcctggggaagagttttggtgagatatttggcttcaatactttgatttcatttcctctagttgaagtatatgggcaaagaacttcggtgaatgttgtcttgttgtgttgtagggactactatatccccttggattgttaccttggatgcgcttgagccttttggttgtcaagctcccaagcaggttggtacttaggcatcacattcggtaccat

SEQ ID NO: 43 - DNA, 32 bp, oligonucleotide
atgaattccatggagtcaaagattcaaataga

SEQ ID NO: 44 - DNA, 32 bp, oligonucleotide
atgaattcggacaatcagtaaattgaacggag

(19) United States
(12) Patent Application Publication
(10) Pub. No.: US 2003/0182679 A1
(43) Pub. Date: Sep. 25, 2003

(54) Improved method for the biosynthesis of vitamin e

(76) Inventors: Michael Geiger, Quedlinburg (DE); Marcus Ebneth, Berlin (DE); Irene Kunze, Gatersleben (DE)

Correspondence Address: NIXON & VANDERHYE, PC, 1100 N GLEBE ROAD, 8TH FLOOR, ARLINGTON, VA, 22201-4714, US

(21) Appl. No.: 10/380,132

(22) PCT Filed: Sep. 18, 2001
(86) PCT No.: PCT/EP01/10779

(30) Foreign Application Priority Data
Sep. 19, 2000 (DE) 100 46 462.9

(51) Int. Cl. A01H 1/00; C12N 15/82; A01N 25/00

(52) U.S. Cl. 800/278; 504/116.1

(57) ABSTRACT

The invention relates to improved processes for the biosynthesis of vitamin E. These processes comprise inhibiting the breakdown of homogentisate via maleyl acetoacetate and fumaryl acetoacetate to give fumarate and acetoacetate. Also in accordance with the invention is the combination of this inhibition with processes which increase the supply of homogentisate, or which promote the conversion of homogentisate into vitamin E.

According to the invention are nucleic acid constructs and vectors with which the processes according to the invention can be carried out, and transgenic plant organisms generated on the basis of this.

The invention relates to improved processes for the biosynthesis of vitamine E. These processes are characterized by inhibiting homogentisate (HG) breakdown via maleyl acetoacetate (MAA), fumaryl acetoacetate (FAA) to give fumarate and acetoacetate. Also in accordance with the invention is the combination of this inhibition with processes which further increase the supply of homogentisate, or which promote the conversion of homogentisate into vitamin E.

Homogentisate is an important metabolite. It is a degradation product of the amino acids tyrosine and phenylalanine. In humans and animals, homogentisate is broken down further to maleyl acetoacetate, subsequently to fumaryl acetoacetate and then into fumarate and acetoacetate. Plants and other photosynthesizing microorganism furthermore utilize homogentisate as starting material for the synthesis of tocopherols and tocotrienols.

The naturally occurring eight compounds with vitamin E activity are derivatives of 6-chromanol (Ullmann's Encyclopedia of Industrial Chemistry, Vol. A 27 (1996), VCH Verlagsgesellschaft, Chapter 4., 478-488, vitamin E). The tocopherol group (1a-d) has a saturated side chain, while the tocotrienol group (2a-d) has an unsaturated side chain:

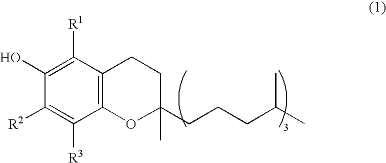

1a, α-tocopherol: R¹═R²═R³═CH₃

1b, β-tocopherol [148-03-8]: R¹═R³ ═CH₃, R²═H

1c, γ-tocopherol [54-28-4]: R¹═H, R²═R³═CH₃

1d, δ-tocopherol [119-13-1]: R¹═R²═H, R³═CH₃

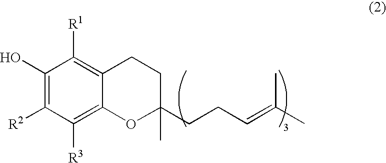

2a, α-tocotrienol [1721-51-3]: R¹═R²═R³═CH₃

2b, β-tocotrienol [490-23-3]: R¹═R³═CH₃, R²═H

2c, γ-tocotrienol [14101-61-2]: R¹═H, R²═R³═CH₃

2d, δ-tocotrienol [25612-59-3]: R¹═R²═H, R³═CH₃

For the purposes of the present invention, vitamin E is to be understood as meaning all of the eight abovementioned tocopherols and tocotrienols with vitamin E activity.

These compounds with vitamin E activity are important natural lipid-soluble antioxidants. Vitamin E deficiency leads to pathophysiological situations in humans and animals. It has been revealed in epidemiological studies that food supplementation with vitamin E reduces the risk of developing cardiovascular diseases or cancer. Furthermore, a positive effect on the immune system and the prevention of general age-related degenerative symptoms have been described (Traber M G, Sies H; Annu Rev Nutr. 1996;16:321-47). The function of vitamin E is probably a stabilization of the biomembranes and a reduction of free radicals as they are formed, for example, upon the lipid oxidation of polyunsaturated fatty acids (PUFAs).

Little work has gone into studying the function of vitamin E in the plants themselves. Possibly, however, it seems to play an important role in the stress response of the plant, in particular oxidative stress. Increased vitamin E levels were linked to improved stability and shelf life of plant-derived products. The supplementation with vitamin E of animal nutrition products has a positive effect on meat quality and the shelf life of the meat and meat products in, for example, pigs, cattle and poultry.

Thus, vitamin E compounds are of great economic value as additives in the food and feed sectors, in pharmaceutical formulations and in cosmetic applications.

In nature, vitamin E is synthesized exclusively by plants and other photosynthetically active organisms (for example cyanobacteria). The vitamin E content varies greatly. Most of the staple food plants (for example wheat, rice, maize, potato) only have a very low vitamin E content (Hess, Vitamin E, α-tocopherol, In Antioxidants in Higher Plants, editors: R. Ascher and J. Hess, 1993, CRC Press, Boca Raton, pp. 111-134). As a rule, oil crops have a markedly higher vitamin E content, with β-, γ- and δ-tocopherol dominating. The recommended daily dose of vitamin E is 15-30 mg.

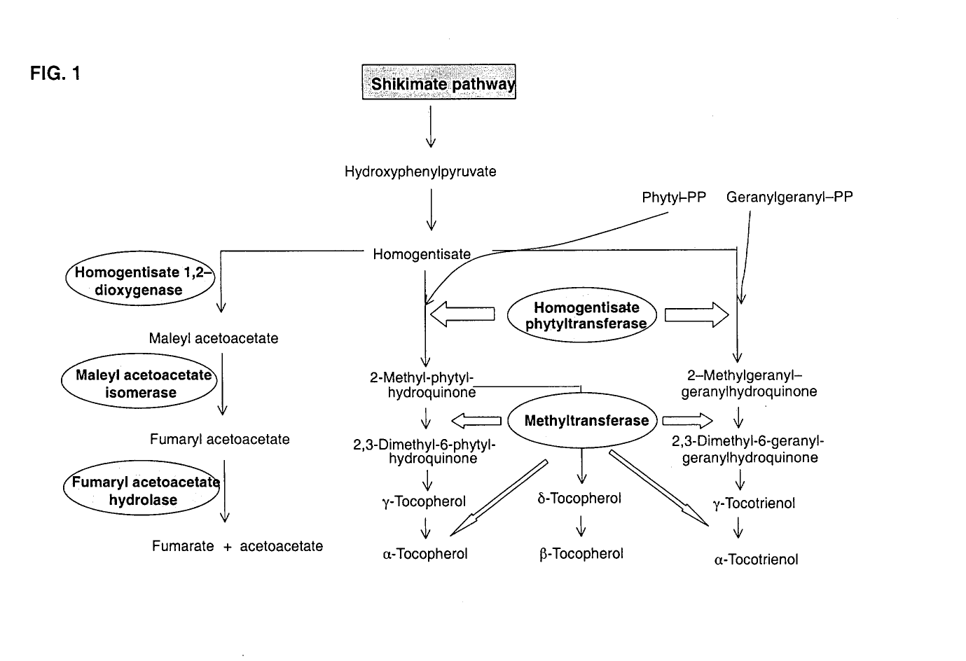

FIG. 1 shows a biosynthetic scheme of tocopherols and tocotrienols.

During biosynthesis, homogentisic acid (homogentisate; HG) is bound to phytyl pyrophosphate (PPP) or geranylgeranyl pyrophosphate in order to form the precursors of α-tocopherol and α-tocotrienol, namely 2-methylphytylhydroquinone and 2-methylgeranylgeranyl hydroquinone, respectively. Methylation steps with S-adenosylmethionine as methyl donor first gives 2,3-dimethyl-6-phytylhydroquinone, cyclization then gives γ-tocopherol, and further methylation gives α-tocopherol. Furthermore, β- and δ-tocopherol can be synthesized by methylation of 2-methylphytylhydroquinone.

Little is known as yet about increasing the metabolite flux to increase the tocopherol or tocotrienol content in transgenic organisms, for example in transgenic plants, by overexpressing individual biosynthesis genes.

WO 97/27285 describes a modification of the tocopherol content by increased expression or by downregulation of the enzyme p-hydroxyphenyl-pyruvate dioxygenase (HPPD).

WO 99/04622 describes gene sequences encoding a γ-tocopherol methyltransferase from Synechocystis PCC6803 and Arabidopsis thaliana, and its incorporation into transgenic plants.

WO 99/23231 demonstrates that the expression of a geranylgeranyl oxidoreductase in transgenic plants results in an increased tocopherol biosynthesis.

WO 00/10380 shows a modification of the vitamin E composition using 2-methyl-6-phytylplastoquinol methyltransferase.

It has been shown by Shintani and DellaPenna that overexpression of γ-tocopherol methyltransferase can markedly increase the vitamin E content (Shintani and Dellapenna, Science 282 (5396):2098-2100, 1998).

All reactions of vitamin E biosynthesis involve homogentisate. As yet, most studies have concentrated on the overexpression of genes of vitamin E or homogentisate biosynthesis (see above). The competing reactions which break down homogentisage and thus remove it from vitamin E biosynthesis have received little attention to date.

The breakdown of homogentisate via maleyl acetoacetate and fumaryl acetoacetate into fumarate and acetoacetate has been described for nonphotosynthetically active organisms, mainly animal organisms (Fernandez-Canon J M et al., Proc Natl Acad Sci USA. 1995; 92 (20):9132-9136). Animal organisms exploit this metabolic pathway for breaking down aromatic amino acids which are predominantly ingested with the food. Its function and relevance in plants, in contrast, is unclear. The reactions are catalyzed by homogentisate 1,2-dioxygenase (HGD; EC No.: 1.13.11.5), maleyl-acetoacetate isomerase (MAAI; EC No.: 5.2.1.2.) and fumaryl acetoacetate hydrolase (FAAH; EC No.: 3.7.1.2).

The Arabidopsis thaliana homogentisate 1,2-dioxygenase (HGD) gene is known (Genbank Acc.-No. AF130845). Owing to a homology with the Emericella nidulans fumaryl acetoacetate hydrolase (gb|L41670), the Arabidopsis thaliana fumaryl acetoacetate hydrolase gene had already been annotated as having similarity to the former (Genbank Acc.-No. AC002131). However, express mention may be made in the relevant Genbank entry that the annotation alone is based on similarity and not on experimental data. The Arabidopsis maleyl-acetoacetate isomerase (MAAI) gene was present in Genbank as a gene (AC005312), but annotated as a putative glutathione S-transferase. An Emericella nidulans MAAI was known (Genbank Acc.-No. EN 1837).

In an abstract (Abstract No. 413) presented at the 1999 Annual Meeting of the American Society of Plant Physiologists (Jul. 24-28, 1999, Baltimore, USA), Tsegaye et al. conjecture an advantage in the combination of a cross of HPPD-overexpressing plants with plants in which HGD is downregulated by an antisense approach.

Despite some success, there continues to exist a demand for optimizing vitamin E biosynthesis.

It is an object of the present invention to provide further processes which influence the vitamin E biosynthetic pathway and thus lead to further advantageous transgenic plants with an elevated vitamin E content.

We have found that this object is achieved by identifying the homogentisate/maleyl acetoacetate/fumaryl acetoacetate/fumarate catabolic pathway as essential competitive pathway for the vitamin E biosynthetic pathway. We have found that inhibition of this catabolic pathway results in an optimization of vitamin E biosynthesis.

Accordingly, the present invention firstly relates to processes for a vitamin E production by reducing the HGD, MAAI and/or FAAH activity. A combination of the above-described inhibition of the homogentisate catabolic pathway with other processes which lead to an improved vitamin E biosynthesis by promoting the conversion of homogentisate into vitamin E proves to be especially advantageous. This can be realized by an increased supply of reactants or by an increased reaction of homogentisate with precisely these reactants. This effect can be achieved for example by overexpressing homogentisate phytyltransferase (HGPT), geranylgeranyl oxidoreductase, 2-methyl-6-phytylplastoquinol methyltransferase or γ-tocopherol methyltransferase.

A combination with genes which promote formation of homogentisate, such as, for example, HPPD or the TyrA gene, is furthermore advantageous.

Inhibition of the catabolic pathway from homogentisate via maleyl acetoacetate and fumaryl acetoacetate to give fumarate and acetatoacetate can be realized in a plurality of ways.

The invention relates to nucleic acid constructs comprising at least one nucleic acid sequence (anti-MAAI/FAAH), which is capable of inhibiting the maleyl acetoacetate/fumaryl acetoacetate/fumarate pathway, or one of its functional equivalents.

The invention furthermore relates to above-described nucleic acid constructs which, besides the anti-MAAI/FAAH nucleic acid sequence, additionally comprise at least one nucleic acid sequence (pro-HG) which is capable of increasing the biosynthesis of homogentisate (HG), or one of its functional equivalents, or at least one nucleic acid sequence (pro-vitamin E) which is capable of increasing vitamin E biosynthesis starting from homogentisate, or one of its functional equivalents, or a combination of pro-HG and pro-vitamin E, or their functional equivalents.

The invention furthermore relates to nucleic acid constructs comprising a nucleic acid sequence (anti-HGD) which is capable of inhibiting homogentisate 1,2-dioxygenase (HGD), or one of its functional equivalents.

The invention furthermore relates to said anti-HGD nucleic acid constructs which, besides the anti-HGD nucleic acid sequence, additionally comprise at least one nucleic acid sequence encoding a bifunctional chorismate mutase/prephenate dehydrogenase (TyrA), or one of its functional equivalents, or at least one nucleic acid sequence (pro-vitamin E), which is capable of increasing vitamin E biosynthesis starting from homogentisate, or one of its function equivalents, or a combination of pro-vitamin E and TyrA sequences, or one of their functional equivalents.

TyrA encodes a bifunctional chorismate mutase/prephenate dehydrogenase from E. coli, a hydroxyphenylpyruvate synthase containing the enzymatic activities of a chorismate mutase and a prephenate dehydrogenase which converts chorismate into hydroxyphenyl pyruvate, the starting material for homogentisate (Christendat D, Turnbull J L. Biochemistry. Apr. 13, 1999;38(15):4782-93; Christopherson R I, Heyde E, Morrison J F. Biochemistry. Mar. 29, 1983;22(7):1650-6.).

The invention furthermore relates to nucleic acid constructs comprising at least one nucleic acid sequence (pro-HG) which is capable of increasing homogentisate (HG) biosynthesis, or one of its functional equivalents, and at least one nucleic acid sequence (pro-vitamin E) which is capable of increasing vitamin E biosynthesis starting from homogentisate, or one of its functional equivalents.

Also in accordance with the invention are functional analogs of the abovementioned nucleic acid constructs. Functional analogs means, in this context, for example a combination of the individual nucleic acid sequences

1. on a polynucleotide (multiple constructs)

2. on several polynucleotides in one cell (cotransformation)

3. by crossing various transgenic plants, each of which comprises at least one of said nucleotide sequences.

The nucleic acid sequences present in the nucleic acid construct are preferably linked functionally to genetic control sequences.

The transformation according to the invention of plants with a pro-HG-encoding construct leads to an increased homogentise formation. An undesirable efflux of this metabolite is avoided by simultaneously transforming with anti-HGD, or anti-MAAI/FAAH, in particular the anti-MAAI construct. Thus, an increased amount of homogentisate is available in the transgenic plant for the formation of vitamin E, for example, tocopherols, via the intermediates methyl-6-phytylquinol and 2,3-dimethylphytylquinol (cf. FIG. 1). Not only pro-HG, but also anti-MAAI/FAAH or anti-HGD, leads to an increased supply of homogentisate for vitamin E biosynthesis. The conversion of homogentisate into vitamin E can be improved by combined transformation with a pro-vitamin-E-encoding construct and further increases the biosynthes of vitamin E.

An “increase” in homogentisate biosynthesis is to be interpreted broadly in this context and encompasses an increased homogentisate (HG) biosynthese activity in the plant or the plant part or tissue transformed with a pro-HG construct according to the invention. A variety of strategies for increasing HG biosynthesis activity are encompassed by the invention. The skilled worker recognizes that a series of different methods is available for influencing HG biosynthesis activity in the desired fashion. The processes described subsequently are to be understood as examples and not by way of limitation.

In the strategy which is preferred in accordance with the invention, a nucleic acid sequence (pro-HG) is used which can be transcribed and translated into a polypeptide which increases HG biosynthesis activity. Examples of such nucleic acid sequences are p-hydroxyphenyl-pyruvate dioxygenase (HPPD) from various organisms, or the bacterial TyrA gene product. In addition to the above-described artificial expression of known genes, it is also possible to increase their activity by mutagenizing the polypeptide sequence. Furthermore, increased transcription and translation of the endogenous genes can be achieved, for example, by using artificial transcription factors of the zinc finger protein type (Beerli R R et al., Proc Natl Acad Sci U S A. 2000; 97 (4):1495-500). These factors attach to the regulatory regions of the endogenous genes and cause expression or repression of the endogenous gene, depending on how the factor is designed.

Especially preferred for pro-HG is the use of nucleic acids which encode polypeptide of SEQ ID NO: 8, 11 or 16, especially preferably nucleic acids with the sequences described by SEQ ID NO: 7, 10 or 15.

The “increase” in vitamin E biosynthesis activity is to be understood in a similar fashion, genes being employed here whose activity promote the conversion of homogentisate into vitamin E (tocopherols, tocotrienols) or whose activity promotes the synthesis of reactants of homogentisate such as, for example, phytyl pyrophosphate or geranylgeranyl pyrophosphate. Examples which may be mentioned are homogentisate-phytyltransferase (HGPT), geranylgeranyl oxidoreduktase, 2-methyl-6-phytylplastoquinol methyltransferase and γ-tocopherol methyltransferase. Especially preferred is the use of nucleic acids which encode polypeptides of SEQ ID NO: 14, 20, 22 or 24, especially preferred are those with the sequences described by SEQ ID NO: 13, 19, 21 or 23.

“Inhibition” is to be interpreted broadly in connection with anti-MAAI/FAAH and/or anti-HGD and encompasses the partial, or essentially complete, repression or blocking of the MAAI/FAAH and/or HGD enzyme activity in the plant or the plant part or tissue transformed with an anti-MAAI/FAAH and/or anti-HGD construct according to the invention, which repression or blocking is based on a variety of mechanisms in terms of cell biology. Inhibition for the purposes of the invention also encompasses a quantitative reduction of active HGD, MAAI or FAAH in the plant up to an essentially complete absence of HGD, MAAI or FAAH protein (i.e. absent detectability of HGD and/or MAAI or FAAH enzyme activity or absent immunological detectability of HGD, MAAI or FAAH).

A variety of strategies for reducing or inhibiting the HGD or MAAI or FAAH activity are encompassed by the invention. The skilled worker recognizes that a series of different methods is available for influencing the HGD or MAAI or FAAH gene expression or enzyme activity in the desired manner.

The strategy which is preferred in accordance with the invention encompasses the use of a nucleic acid sequence (anti-MAAI/FAAH and/or anti-HGD) which can be transcribed into an antisense nucleic acid sequence which is capable of inhibiting the HGD or MAAI/FAAH activity, for example by inhibiting the expression of endogenous HGD and/or MAAI or FAAH.

The anti-HGD and/or anti-MAAI/FAAH nucleic acid sequences according to the invention can, in a preferred embodiment, contain the coding nucleic acid sequence of HGD (anti-HGD) and/or MAAI or FAAH (anti-MAAI/FAAH) inserted in antisense orientation, or functional equivalent fragments of the sequences in question.

Especially preferred anti-HGD nucleic acid sequences encompass nucleic acid sequences which encode polypeptides comprising an amino acid sequence of SEQ ID NO: 3 or functional equivalents thereof. Especially preferred are nucleic acid sequences of SEQ ID NO: 1, 2 or 12 or functional equivalents thereof.

Especially preferred anti-MAAI/FAAH nucleic acid sequences encompass nucleic acid sequences which encode polypeptides comprising an amino acid sequence of SEQ ID NO: 5 and 18 or functional equivalents thereof. Especially preferred are nucleic acid sequences of SEQ ID NO: 4, 6, 9 or 17 or functional equivalents thereof, very especially preferred are the part-sequences shown in SEQ ID NO: 41 or 42, or their functional equivalents.

A preferred embodiment of the nucleic acid sequences according to the invention encompasses an HGD, MAAI or FAAH sequence motif of SEQ ID NO: 1, 2, 4, 6, 9, 12, 17, 41 or 42 in antisense orientation. This leads to an increased transcription of nucleic acid sequences in the transgenic plant which are complementary to the endogenous coding HGD, MAAI or FAAH sequence or a part thereof and which hybridize with this sequence at the DNA or RNA level.

The antisense strategy can advantageously be combined with a ribozyme method. Ribozymes are catalytically active RNA sequences which, coupled to the antisense sequences, catalytically cleave the target sequences (Tanner N K. FEMS Microbiol Rev. 1999; 23 (3):257-75). This can increase the efficacy of an anti-sense strategy.

Further methods for inhibiting HGD and/or MAAI/FAAH expression encompass the overexpression of homologous HGD and/or MAAI/FAAH nucleic acid sequences, which leads to cosuppression (Jorgensen et al., Plant Mol. Biol. 1996, 31 (5):957-973), induction of the specific RNA breakdown by the plant with the aid of a viral expression system (amplicon) (Angell, S M et al., Plant J. 1999, 20(3):357-362). These methods are also termed “post-transcriptional gene silencing” (PTGS).

Further methods are the introduction of nonsense mutations into the endogene by means of introducing RNA/DNA oligonucleotides into the plant (Zhu et al., Nat. Biotechnol. 2000, 18(5):555-558) or the generation of knockout mutants with the aid of, for example, T-DNA mutagenesis (Koncz et al., Plant Mol. Biol. 1992, 20(5):963-976) or homologous recombination (Hohn, B. and Puchta, H, Proc. Natl. Acad. Sci. USA. 1999, 96:8321-8323.).

Furthermore, overexpression or repression of genes is also possible using specific DNA-binding factors, for example the abovementioned factors of the zinc finger transcription factor type. Furthermore, factors may be introduced into a cell which inhibit the target protein itself. The protein-binding factors can be, for example, aptamers (Famulok M, and Mayer G. Curr Top Microbiol Immunol. 1999; 243:123-36).

The above-described publications and the methods disclosed therein for regulating plant gene expression are herewith expressly referred to.

An anti-HGD and/or anti-MAAI/FAAH sequence for the purposes of the present invention is thus selected in particular from among:

a) antisense nucleic acid sequences;

b) antisense nucleic acid sequences combined with a ribozyme method

c) nucleic acid sequences encoding homologous HGD and/or MAAI/FAAH and leading to cosuppresion;

d) viral nucleic acid sequences and expression constructs causing HGD and/or MAAI/FAAH-RNA breakdown;

e) nonsense mutants of endogenous HGD- or MAAI/FAAH-encoding nucleic acid sequences;

f) nucleic acid sequences encoding knockout mutants;

g) nucleic acid sequences which are suitable for homologous recombination;

h) nucleic acid sequences encoding specific DNA- or protein-binding factors with anti-HGD and/or anti-MAAI/FAAH activity;

it being possible for the expression of each individual of these anti-HGD or anti-MAAI/FAAH sequences to cause “inhibition” of the HGD and/or MAAI/FAAH activity as defined for the invention. A combined use of such sequences is also feasible.

A nucleic acid construct or nucleic acid sequence is to be understood as meaning in accordance with the invention for example a genomic or a complementary DNA sequence or an RNA sequence and semisynthetic or fully synthetic analogs thereof.

These sequences can exist in linear or circular form, extrachromosomally or integrated into the genome. The pro-HG, pro-vitamin E, anti-HGD or anti-MAAI/FAAH nucleotide sequences of the constructs according to the invention can be generated synthetically or obtained naturally or comprise a mixture of synthetic or natural DNA constituents and can be composed of various heterologous HGD, MAAI/FAAH, pro-HG or pro-vitamin E gene segments of various organisms. The anti-HGD and/or anti-MAAI/FAAH sequence can be derived from one or more exons or introns, in particular exons of the HGD, MAAI or FAAH genes.

Also suitable are artificial nucleic acid sequences as long as they mediate the desired property, for example the increase in the vitamin E content in the plant, by overexpression of at least one pro-HG and/or pro-vitamin E gene and/or expression of an anti-HDG and/or MAAI/FAAH sequence in crop plants, as described above. For example, synthetic nucleotide sequences can be generated which have codons which are preferred by the plants to be transformed. These codons which are preferred by plants can be determined in the customary manner from codons with the highest protein frequency by referring to the codon usage. Such artificial nucleotide sequences can be determined, for example, by backtranslating proteins with HGD and/or MAAI/FAAH and/or pro-HG activity or pro-vitamin E activity which have been constructed by means of molecular modeling, or else by in-vitro selection. Especially suitable are coding nucleotide sequences which have been obtained by backtranslating a polypeptide sequence in accordance with the codon usage which is specific for the host plant. For example, to avoid undesired regulatory mechanisms of the plant, DNA fragments can be backtranslated starting from the amino acid sequence of a bacterial pro-HG, for example the bacterial TyrA gene, taking into consideration the codon usage of the plant, and the complete exogenous pro-HG sequence can be generated therefrom for use in the plant. This is used to express a pro-HG enzyme which is not, or only insufficiently, subject to regulation by the plant, thus allowing full overexpression of the enzyme activity.

All the abovementioned nucleotide sequences can be prepared in a manner known per se by chemical synthesis starting from the nucleotide units, for example by fragment condensation of individual overlapping complementary nucletic acid units of the double helix. Oligonucleotides can be synthesized chemically for example in a known manner by the phosphoamidite method (Voet, Voet, 2nd Edition, Wiley Press New York, page 896-897). When preparing a nucleic acid construct, various DNA fragments can be manipulated in such a way that a nucleotide sequence is obtained which reads in the correct direction and which has a correct reading frame. To connect the nucleic acid fragments to each other, adaptors or linkers can be added to the fragments. The addition of synthetic oligonucleotides and filling in gaps with the aid of the Klenow fragment of DNA polymerase and ligation reactions and general cloning methods are described in Sambrook et al. (1989), Molecular cloning: A laboratory manual, Cold Spring Harbor Laboratory Press.

Functional equivalents of the pro-HG or pro-vitamin E sequences are those sequences which, despite a deviating nucleotide sequence, still encode a protein with the functions desired in accordance with the invention, i.e. an enzyme whose activity directly or indirectly increases the formation of homogentisate (pro-HG), or an enzyme whose activity directly or indirectly promotes the conversion of homogentisate to vitamin E (pro-vitamin E).

Functional equivalents of anti-HGD and/or anti-MAAI/FAAH encompass those nucleotide sequences which sufficiently repress the HGD and/or MAAI/FAAH enzyme functions in the transgenic plant. This can be effected for example by preventing or repressing HGD and/or MAAI/FAAH processing, the transport of HGD and/or MAAI/FAAH or their mRNA, inhibiting ribosome attachment, inhibiting RNA splicing, inducing an RNA-degrading enzyme and/or inhibiting translational elongation or termination. Direct repression of the endogenous genes by DNA-binding factors, for example of the zinc finger transcription factor type, is furthermore possible. Direct inhibition of the polypeptides in question, for example by aptamers, is also possible. Various examples are given hereinabove.

Functional equivalents are also to be understood as meaning, in particular, natural or artificial mutations of an originally isolated sequence encoding HGD and/or MAAI/FAAH or pro-HG or pro-vitamin E which continue to show the desired function. Mutations encompass substitutions, additions, deletions, exchanges or insertions of one or more nucleotide residues. Thus, the present invention also encompasses, for example, those nucleotide sequences which are obtained by modifying the HGD and/or MAAI/FAAH and/or pro-HG or pro-vitamin E nucleotide sequence. The purpose of such a modification may be, for example, the further limitation of the coding sequence contained therein or else, for example, the insertion of further restriction enzyme cleavage sites or the removal of superfluous DNA.

Techniques known per se, such as in-vitro mutagenesis, primer repair, restriction or ligation may be used in cases where insertions, deletions or substitutions such as, for example, transitions and transversions, are suitable. Complementary ends of the fragments may be provided for ligation by manipulations such as, for example, restrictions, chewing-back or filling in overhangs for blunt ends.

Substitution is to be understood as meaning the exchange of one or more amino acids for one or more amino acids. Exchanges which are preferably carried out are so-called conservative exchanges where the replaced amino acid has a similar property as the original amino acid, for example the exchange of Glu for Asp, Gln for Asn, Val for Ile, Leu for Ile and Ser for Thr.

Deletion is the replacement of an amino acid by a direct bond. Preferred positions for deletions are the termini of the polypeptide and the linkages between the individual protein domains.

Insertions are introductions of amino acids into the polypeptide chain, a direct bond being formally replaced by one or more amino acids.

Homology between two proteins is understood as meaning the identity of the amino acids over in each case the entire length of the protein which is calculated by comparison with the aid of the program algorithm GAP (UWGCG, University of Wisconsin, Genetic Computer Group) setting the following parameters:

Gap Weight: 12

Length Weight: 4

Average Match: 2.912

Average Mismatch: −2.003

Accordingly, a sequence which has at least 20% homology of the nucleic acid level with the sequence SEQ ID NO. 6 is to be understood as meaning a sequence which, upon comparison of its sequence with the sequence SEQ ID NO. 6 using the above program algorithm with the above parameter set, has at least 20% homology.

Functional equivalents derived from one of the nucleic acid sequences used in the nucleic acid constructs or vectors according to the invention, for example by substitution, insertion or deletion of amino acids or nucleotides, have at least 20% homology, preferably 40% homology, by preference at least 60% homology, preferably at least 80% homology, especially preferably at least 90% homology.

Further examples for the nucleic acid sequences employed in the nucleic acid constructs or vectors according to the invention can be found readily from various organisms whose genomic sequence is known, such as, for example, Arabidopsis thaliana, by homology alignments of the amino acid sequences or from the corresponding backtranslated nucleic acid sequences from databases.

Functional equivalents also encompass those variants whose function is reduced or increased compared to the starting gene or gene fragment, i.e., for example, those pro-HG or pro-vitamin E genes which encode a polypeptide variant with a lower or higher enzymatic activity than that of the original gene.

Further suitable functionally equivalent nucleic acid sequences which may be mentioned are sequences which encode fusion proteins, part of the fusion protein being, for example, a pro-HG or pro-vitamin E polypeptide or a functionally equivalent portion thereof. The second portion of the fusion protein can be, for example, a further polypeptide with enzymatic activity (for example a further pro-HG or pro-vitamin E polypeptide or a functionally equivalent portion thereof) or an antigenic polypeptide sequence with the aid of which pro-HG or pro-vitamin E expression can be detected (for example Myc tag or His tag). However, they are preferably a regulatory protein sequence such as, for example, a signal or transit peptide which leads the pro-HG or pro-vitamin E protein to the desired site of action.

The invention furthermore relates to recombinant vectors comprising at least one nucleic acid construct in accordance with the above definition, a nucleic acid sequence encoding an HGD, MAAI or FAAH, or combinations of these options.

The nucleic acid sequences or nucleic acid constructs present in the vectors are preferably linked functionally to genetic control sequences.

Examples of vectors according to the invention may encompass expression constructs of the following type:

a) 5′-plant-specific promoter/anti-HGD/terminator-3′

b) 5′-plant-specific promoter/anti-MAAI/FAAH/terminator-3′

c) 5′-plant-specific promoter/pro-HG/terminator-3′

d) 5′-plant-specific promoter/pro-vitamin E/terminator-3′

The invention also expressly relates to vectors which are capable of expressing polypeptides with an HGD, MAAI or FAAH activity. The sequences encoding these genes are preferably derived from plants, cyanobacteria, mosses, fungi or algae. The sequences encoding polypeptides of SEQ ID NO: 3, 5 and 18 are especially preferred.

In this context, the coding pro-HG or pro-vitamin E sequence, and the sequences for the expression of polypeptides with HGD, MAAI or FAAH activity, may also be replaced by a coding sequence for a fusion protein of transit peptide and the sequence in question.

Preferred examples encompass vectors and may comprise one of the following expression constructs:

a) 5′-35S promoter/anti-MAAI/FAAH/OCS terminator-3′

b) 5′-35S promoter/anti-HGD/OCS terminator-3′;

c) 5′-legumin B promoter/pro-HG/NOS terminator-3′

d) 5′-legumin B promoter/pro-vitamin E/NOS-terminater-3 ′

e) 5′-legumin B promoter/HGD/NOS terminator-3′

f) 5′-legumin B promoter/MAAI/NOS terminator-3′

g) 5′-legumin B promoter/FAAH/NOS terminator-3′

In this context, too, the coding pro-HG sequence or pro-vitamin E sequence may also be replaced by a coding sequence for a fusion protein of transit peptide and pro-HG or pro-vitamin E.

A cotransformation with more than one of the abovementioned examples a.) to g.) may be required for the advantageous processes according to the invention for optimizing vitamin E biosynthesis. Furthermore, transformation with one or more vectors, each of which comprises a combination of the abovementioned constructs, may be advantageous. Preferred examples encompass vectors comprising the following constructs:

a) 5′-35S promoter/anti-MAAI/FAAH/OCS terminator/legumin B promoter/pro-HG/NOS terminator-3′;

b) 5′-35S promoter/anti-MAAI/FAAH/OCS terminator/legumin B promoter/pro-vitamin E/NOS terminator-3′;

c) 5′-35S promoter/anti-HGD/OCS terminator/legumin B promoter/pro-vitamin E/NOS terminator-3′;

d) 5′-35S promoter/pro-HG/OCS terminator/legumin B promoter/pro-vitamin E/NOS terminator-3′;

Constructs a) to d) permit the simultaneous transformation of the plant with pro-HG and/or pro-vitamin E and anti-HGD and/or anti-MAAI/FAAH .

Using the above-cited recombination and cloning techniques, the nucleic acid constructs can be cloned into suitable vectors which make possible the amplification, for example in E. coli. Suitable cloning vectors are, inter alia, pBR332, pUC series, M13mp series and pACYC184. Especially suitable are binary vectors which are capable of replicating both in E. coli and in agrobacteria.

The nucleic acid constructs according to the invention are preferably inserted into suitable transformation vectors. Suitable vectors are described, inter alia, in Methods in Plant Molecular Biology and Biotechnology (CRC Press), Chapter 6/7, pp. 71-119 (1993). They are preferably cloned into a vector such as, for example, pBin19, pBinAR, pPZP200 or pPTV, which is suitable for transforming Agrobacterium tumefaciens. The agrobacteria transformed with such a vector can then be used in the known manner for transforming plants, in particular crop plants such as, for example, oilseed rape, for example by bathing scarified leaves or leaf sections in an agrobacterial solution and subsequently culturing them in suitable media. The transformation of plants by agrobacteria is known, inter alia, from F. F. White, Vectors for Gene Transfer in Higher Plants; in Transgenic Plants, Vol. 1, Engineering and Utilization, edited by S. D. Kung and R. Wu, Academic Press, 1993, pp. 15-38. Transgenic plants which comprise the above-described nucleic acid constructs integrated can be regenerated from the transformed cells of the scarified leaves or leaf sections in the known manner.

The nucleic acid sequences present in the nucleic acid constructs and vectors according to the invention can be linked functionally to at least one genetic control sequence. Genetic control sequences ensure for example transcription and translation in prorokaryotic or eukaryotic organisms. The constructs according to the invention preferably comprise, 5′-upstream of the coding sequence in question, a promoter and 3′-downstream a terminator sequence and, if appropriate, other customary regulatory elements, in each case functionally linked to the coding sequence. Functional linkage is to be understood as meaning, for example, the sequential arrangement of promoter, coding sequence, terminator and, if appropriate, further regulatory elements in such a way that each of the regulatory elements can fulfill its intended function upon expression of the coding sequence or the antisense sequence. This does not necessarily require direct linkage in the chemical sense. Genetic control sequences such as, for example, enhancer sequences, can also exert their function from other DNA molecules toward the target sequence.

Examples are sequences to which inductors or repressors bind, thus regulating the expression of the nucleic acid. In addition to these novel control sequences, or instead of these sequences, the natural regulation of these sequences before the actual structural genes may still be present and, if appropriate, may have been modified genetically so that the natural regulation has been switched off and expression of the genes has been increased. However, the nucleic acid construct may also have a simpler structure, that is to say no additional regulatory signals are inserted before the abovementioned genes, and the natural promoter with its regulation is not removed. Instead, the natural control sequence is mutated in such a way that regulation no longer takes place and gene expression is enhanced. These modified promoters may also be placed before the natural genes by themselves in order to increase the activity.

Moreover, the nucleic acid construct may advantageously comprise one or more enhancer sequences linked functionally to the promoter, and these make possible an increased expression of the nucleic acid sequence. At the 3′end of the DNA sequences, too, additional advantageous sequences may be inserted, such as further regulatory elements or terminators. The genes mentioned hereinabove may be present in the gene construct in the form of one or more copies.

Additional sequences which are preferred for functional linkage, but not limited thereto, are further targeting sequences which differ from the transit-peptide-encoding sequences and which ensure subcellular localization in the apoplasts, in the vacuole, in plastids, in the mitochondrion, in the endoplasmatic reticulum (ER), in the nucleus, in eleoplasts or other compartments; and translation enhancers such as the tobacco mosaic virus 5′leader sequence (Gallie et al., Nucl. Acids Res. 15 (1987), 8693-8711), and the like.

Control sequences are furthermore to be understood as those sequences which make possible homologous or heterologous recombination and/or insertion into the genome of a host organism, or which permit the removal from the genome. In the case of homologous recombination, the endogenous gene may be inactivated fully, for example. Furthermore, it may be exchanged for a synthetic gene with increased and modified activity. Methods such as the cre/lox technology permit tissue-specific, in some cases inducible, removal of the target gene from the genome of the host organism (Sauer B. Methods. 1998; 14(4):381-92). This involves adding certain flanking sequences (lox sequences) to the target gene, which later make possible removal by means of cre recombinase.

Various control sequences are suitable, depending on the host organism or starting organism described in greater detail hereinbelow which is transformed into a genetically modified or transgenic organism by introducing the nucleic acid constructs.

Advantageous control sequences for the nucleic acid constructs according to the invention, for the vectors according to the invention, for the process according to the invention for the preparation of vitamin E and for the genetically modified organisms described hereinbelow are present, for example, in promoters such as cos, tac, trp, tet, lpp, lac, lpp-lac, laciq, T7, T5, T3, gal, trc, ara, SP6, 1-PR or in the 1-PL promoter, all of which are advantageously used Gram-negative bacteria.

Further advantageous control sequences are present, for example, in the Gram-positive promoters amy and SPO2, in the yeast or fungal promoters ADC1, MFa, AC, P-60, CYC1, GAPDH, TEF, rp28, ADH or in the plant promoters CaMV/35S [Franck et al., Cell 21(1980) 285-294], PRP1 [Ward et al., Plant. Mol. Biol. 22 (1993)], SSU, OCS, LEB4, USP, STLS1, B33, NOS; FBPaseP (WO 98/18940) or in the ubiquitin or phaseolin promoter.

A preferred promoter for the nucleic acid constructs is, in principle, any promoter which is capable of governing the expression of genes, in particular foreign genes, in plants. A promoter which is preferably used is, in particular, a plant promoter or a promoter derived from a plant virus. Especially preferred is the cauliflower mosaic virus CaMV 35S promoter (Franck et al., Cell 21 (1980), 285-294). As is known, this promoter comprises various recognition sequences for transcriptional effectors which, in their totality, lead to permanent and constitutive expression of the gene which has been inserted (Benfey et al., EMBO J. 8 (1989), 2195-2202). A further example of a suitable promoter is the legumin B promoter (accession No. X03677).

The nucleic acid constructs may also comprise a chemically inducible promoter by means of which expression of the exogenous gene in the plant can be governed at a particular point in time. Such promoters, such as, for example, the PRP1 promoter (Ward et al., Plant. Mol. Biol. 22 (1993), 361-366), a salicylic-acid-inducible promoter (WO 95/19443), a benzenesulfonamide-inducible promoter (EP-A-0388186), a tetracyclin-inducible promoter (Gatz et al., (1992) Plant J. 2, 397404), an abscisic-acid-inducible promoter (EP-A 335528) or an ethanol- or cyclohexanone-inducible promoter (WO 93/21334) may also be used.

Furthermore, particularly preferred promoters are those which ensure expression in tissues or plant parts in which the biosynthesis of vitamin E or its precursors takes place or in which the products are advantageously accumulated. Promoters which must be mentioned in particular are those for the entire plant owing to constitutive expression, such as, for example, the CaMV promoter, the Agrobacterium OCS promoter (octopine synthase), the Agrobacterium NOS promoter (nopaline synthase), the ubiquitin promoter, promoters of vacuolar ATPase subunits, or the promoter of a prolin-rich protein from wheat (WO 91/13991). Promoters which must be mentioned in particular are those which ensure leaf-specific expression. Promoters which must be mentioned are the potato cytosolic FBPase promoter (WO 97/05900), the Rubisco (ribulose-1,5-bisphosphate carboxylase) SSU (small subunit) promoter, or the potato ST-LSI promoter (Stockhaus et al., EMBO J. 8 (1989), 244-245). Examples of seed-specific promoters are the phaseolin promoter (U.S. Pat. No. 5,504,200), the USP promoter (Baumlein, H. et al., Mol. Gen. Genet. (1991) 225 (3), 459-467) or the LEB4 promoter (Fiedler, U. et al., Biotechnology (NY) (1995), 13 (10) 1090) together with the LEB4 signal peptide.

Examples of other suitable promoters are specific promoters for tubers, storage roots or roots, such as, for example, the patatin promoter class I (B33), the potato cathepsin D inhibitor promoter, the starch synthase (GBSS1) promoter or the sporamin promoter, fruit-specific promoters such as, for example, the tomato fruit-specific promoter (EP-A 409625), fruit-maturation-specific promoters such as, for example, the tomato fruit-maturation-specific promoter (WO 94/21794), flower-specific promoters such as, for example, the phytoene synthase promoter (WO 92/16635) or the promoter of the P-rr gene (WO 98/22593) or specific plastid or chromoplast promoters such as, for example, the RNA polymerase promoter (WO 97/06250) or else the Glycine max phosphoribosyl pyrophosphate amidotransferase promoter (see also Genbank Accession Number U87999) or another node-specific promoter such as in EP-A 249676.

In principle, all natural promoters together with their regulatory sequences such as those mentioned above can be used for the process according to the invention. In addition, synthetic promoters can also be used advantageously.

Polyadenylation signals which are suitable as control sequences are plant polyadenylation signals, preferably those which essentially correspond to Agrobacterium tumefaciens T-DNA polyadenylation signals, in particular to gene 3 of the T-DNA (octopine synthase) of the Ti plasmid pTiACHS (Gielen et al., EMBO J. 3 (1984), 835 et seq.) or functional equivalents thereof. Examples of particularly suitable terminator sequences are the OCS (octopine synthase) terminator and the NOS (nopaline synthase) terminator.

A nucleic acid construct is generated, for example, by fusing a suitable promoter to a suitable anti-HGD, anti-MAAI/FAAH, pro-HG, pro-vitamin E, HGD, MAAI or FAAH nucleotide sequence, if appropriate a sequence encoding a transit peptide, preferably a chloroplast-specific transit peptide, which sequence is preferably arranged between the promoter and the nucleotide sequence in question, and a terminator or polyadenylation signal. To do this, customary recombination and cloning techniques are used as they are described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

As already mentioned, it is also possible to use nucleic acid constructs whose DNA sequence encodes a pro-HG, pro-vitamin E, HGD, MAAI or FAAH fusion protein, a portion of the fusion protein being a transit peptide which governs the translocation of the polypeptide. The following may be mentioned by way of example: chloroplast-specific transit peptides which are eliminated enzymatically after translocation into the chloroplasts.

The pro-HG, pro-vitamin E, HGD, MAAI or FAAH nucleotide sequences are preferably linked functionally to the coding sequence of a plant organell-specific transit peptide. The transit peptide preferably has specificity for individual cell compartments of the plant, for example the plastids, such as, for example, the chloroplasts, chromoplasts and/or leukoplasts. The transit peptide guides the polypeptides which have been expressed to the desired target in the plant and, once the target is reached, is eliminated, preferably proteolytically. In the expression construct according to the invention, the coding transit peptide sequence is preferably located 5′-upstream of the coding pro-HG, pro-vitamin E, HGD, MAAI or FAAH sequence. A transit peptide which must be mentioned in particular is the transit peptide which is derived from the plastid Nicotiana tabacum transketolase (TK) or a functional equivalent of this transit peptide (for example the transit peptide of the RubisCO small subunit, or of ferredoxin:NADP oxidoreductase or else isopentenyl pyrophosphate isomerase-2).

The invention furthermore relates to transgenic organisms transformed with at least one nucleic acid construct according to the invention or a vector according to the invention, and to cells, cell cultures, tissues, parts—such as, for example, leaves, roots and the like in the case of plant organisms—or propagation material derived from such organisms.

Organisms, starting organisms or host organisms are to be understood as meaning prokaryotic or eukaryotic organisms such as, for example, microorganisms or plant organisms. Preferred microorganisms are bacteria, yeasts, algae or fungi.

Preferred bacteria are bacteria of the genus Escherichia, Erwinia, Agrobacterium, Flavobacterium, Alcaligenes or cyanobacteria, for example, of the genus Synechocystis.

Preferred microorganisms are, above all, those which are capable of infecting plants and thus of transferring the constructs according to the invention. Preferred microorganisms are those from among the genus Agrobacterium and, in particular, the species Agrobacterium tumefaciens.

Preferred yeasts are Candida, Saccharomyces, Hansenula or Pichia. plant organisms are, for the purposes of the invention, monocotyledonous and dicotyledonous plants. The trasngenic plants according to the invention are selected in particular from among monocotyledonous crop plants such as, for example, cereals such as wheat, barley, sorghum and millet, rye, triticale, maize, rice or oats, and sugar cane. The transgenic plants according to the invention are furthermore selected in particular from among dicotyledonous crop plants such as, for example,

Brassicaceae such as oilseed rape, cress, Arabidopsis, cabbages or canola,

Leguminosae such as soybean, alfalfa, pea, bean plants or peanut Solanaceae such as potato, tobacco, tomato, aubergine or bell pepper,

Asteraceae such as sunflower, Tagetes, lettuce or calendula,

Cucurbitaceae such as melon, pumpkin or zucchini, and also linseed, cotton, hemp, flax, red pepper, carrot, sugar beet and the various tree, nut and grapevine species.

Especially preferred are Arabodopsis thaliana, Nicotiana tabacum, Tagetes erecta, Calendula vulgaris and all genera and species which are suitable for the production of oils, such as oil crops (such as, for example, oilseed rape), nut species, soybean, sunflower, pumpkin and peanut.

Plant organisms for the purposes of the invention are, furthermore, further photosynthetically active organisms, or organisms which are capable of synthesizing vitamin E, such as, for example, algae or cyanobacteria, and also mosses.

Preferred algae are green algase, such as, for example, algae of the genus Haematococcus, Phaedactylum tricornatum, Volvox or Dunaliella.

The transfer of foreign genes into the genome of an organism, for example a plant, is termed transformation. It exploits the above-described methods of transforming and regenerating plants from plant tissues or plant cells for transient or stable transformation. Suitable methods are protoplast transformation by polyethylene glycol-induced DNA uptake, the biolistic method using the gene gun, the particle bombardment method, electroporation, incubation of dry embryos in DNA-containing solution, microinjection and agrobacterium-mediated gene transfer. The abovementioned methods are described, for example, in B. Jenes et al., Techniques for Gene Transfer, in: Transgenic Plants, Vol. 1, Engineering and Utilization, edited by S. D. Kung and R. Wu, Academic Press (1993), 128-143 and in Potrykus, Annu. Rev. Plant Physiol. Plant Molec. Biol. 42 (1991), 205-225). The construct to be expressed is preferably cloned into a vector which is suitable for transforming Agrobacterium tumefaciens for example pBin19 (Bevan et al., Nucl. Acids Res. 12 (1984) , 8711).

The expression efficacy of the recombinantly expressed nucleic acids can be determined, for example, in vitro by shoot-meristem propagation. In addition, changes in the nature and level of the expression of the pro-HG or pro-vitamin E genes and their effect on vitamin E biosynthesis performance, can be tested on test plants in greenhouse experiments.

The invention furthermore relates to transgenic organisms as described above whose vitamin E production is improved in comparison with the untransformed wild type.

In accordance with the invention are furthermore cells, cell cultures, parts—such as, for example, roots, leaves etc. in the case of transgenic plant organisms—, transgenic propagation material, seeds or fruit derived from the above-described transgenic organisms.

Improved vitamin E production means for the purposes of the present invention for example the artificially acquired ability of an increased biosynthesis performance of at least one compound from the group of the tocopherols and tocotrienols in the transgenic organism in comparison with the non-genetically modified starting organism for the duration of at least one plant generation. Preferably, the vitamin E production in the transgenic organism in comparison with the non-genetically modified starting organism, is increased by 10%, especially preferably by 50%, very especially preferably by 100%. The term improved may also mean an advantageously modified qualitative composition of the vitamin E mixture.

The biosynthesis site of vitamin E is, generally, the leaf tissue, but also the seed, so that leaf-specific or seed-specific expression of, in particular, pro-HG and pro-vitamin E sequences and, if appropriate, anti-HGD and/or anti-MAAI/FAAH sequences is meaningful. However, it is obvious that vitamin E biosynthesis need not be restricted to the seed, but can also take place in a tissue-specific manner in all remaining parts of the plant. In addition, constitutive expression of the exogenous gene is advantageous. On the other hand, inducible expression may also be desirable.

Finally, the invention furthermore relates to a process for the production of vitamin E, which comprises isolating the desired vitamin E in a manner known per se from a culture of a plant organism which has been transformed in accordance with the invention.

Genetically modified plants according to the invention with an increased vitamin E content which can be consumed by humans and animals can also be used as foodstuffs or feed, for example directly or following processing, which is known per se.

The invention furthermore relates to the use of polypeptides which encode an HGD, MAAI or FAAH, of the genes and cDNAs on which they are based, and/or of the nucleic acid constructs according to the invention, vectors according to the invention or organisms according to the invention which are derived from them for producing antibodies, protein-binding or DNA-binding factors.

The biosynthetic pathway of the HGD-MAAI-FAAH catabolic pathway offers target enzymes for the development of inhibitors. Therefore, the invention also relates to the use of polypeptides which encode an HGD, MAAI or FAAH, of the genes and cDNAs on which they are based, and/or of the nucleic acid constructs according to the invention, vectors according to the invention or organisms according to the invention which are derived from them as target for finding inhibitors of HGD, MAAI or FAAH.

To be able to find efficient HGD, MAAI or FAAH inhibitors, it is necessary to provide suitable assay systems with which inhibitor-enzyme binding studies can be carried out. To this end, for example, the complete cDNA sequence of HGD, MAAI or FAAH is cloned into an expression vector (for example pQE, Qiagen) and overexpressed in E. coli. The HGD, MAAI or FAAH proteins are particularly suitable for finding HGD-, MAAI- or FAAH-specific inhibitors.

Accordingly, the invention relates to a process for finding inhibitors of HGD, MAAI or FAAH using the abovementioned polypeptides, nucleic acids, vectors or transgenic organisms, which comprises measuring the enzymatic activity of HGD, MAAI or FAAH in the presence of a chemical compound and, if the enzymatic activity is reduced in comparison with the unhibited activity, the chemical compound constitutes an inhibitor. To this end, HGD, MAAI or FAAH can be employed, for example, in an enzyme assay in which the activity of HGD, MAAI or FAAH is determined in the presence and absence of the active ingredient to be assayed. Qualitative and quantitative findings on the inhibitory behavior of the active ingredient to be assayed can be deduced by comparing the two activity determinations. A multiplicity of chemical compounds can be tested in a simple and rapid fashion for herbicidal properties with the aid of the assay system according to the invention. The method allows reproducibly to select, from a large number of substances, specifically those which are very potent in order to subject these substances subsequently to further, in-depth tests with which the skilled worker is familiar.

The inhibitors of HGD, MAAI or FAAH are suitable for functionally increasing vitamin E biosynthesis similarly to the above-described anti-HGD and/or anti-MAAI/FAAH nucleic acid sequences. The invention therefore furthermore relates to processes for improving the vitamin E production using inhibitors of HGD, MAAI or FAAH. The improved production of vitamin E can have a positive effect on the plant since these compounds have an important function in the protection from harmful environmental factors (sun rays, free-radical oxygen). An increased vitamin E production can thus act as growth promoter. The invention therefore furthermore relates to the use of inhibitors of HGD, MAAI or FAAH, obtainable by the above-described process, as growth regulators.

| | |
|---|---|
| Sequences | |
| SEQ ID NO. 1: | Arabidopsis thaliana homogentisate 1,2-dioxygenase (HGD) gene |
| SEQ ID NO. 2: | Arabidopsis thaliana homogentisate 1,2-dioxygenase (HGD) cDNA |
| SEQ ID NO. 3: | Arabidopsis thaliana homogentisate 1,2-dioxygenase (HGD) polypeptide |
| SEQ ID NO. 4: | Arabidopsis thaliana furnaryl acetoacetate hydrolase (FAAH) cDNA |
| SEQ ID NO. 5: | Arabidopsis thaliana fumaryl acetoacetate hydrolase (FAAH) polypeptide |
| SEQ ID NO. 6: | Arabidopsis thaliana maleyl-acetoacetate isomerase (MAAI) gene |
| SEQ ID NO. 7: | TyrA gene encoding a bifunctional chorismate mutase/prephenate |
| | dehydrogenase |
| SEQ ID NO. 8: | TyrA polypeptide encoding a bifunctional chorismate mutase/prephenate |
| | dehydrogenase |
| SEQ ID NO. 9: | Arabidopsis thaliana furnaryl acetoacetate hydrolase (FAAH) gene |
| SEQ ID NO. 10: | Arabidopsis thaliana hydroxyphenyl-pyruvate dioxygenase (HPPD) cDNA |
| SEQ ID NO. 11: | Arabidopsis thaliana hydroxyphenyl-pyruvate dioxygenase (HPPD) polypeptide |
| SEQ ID NO. 12: | Brassica napus homogentisate 1,2-dioxygenase (HGD) cDNA fragment |
| SEQ ID NO. 13: | Synechocystis PCC6803 homogentisate phythyltransferase cDNA |
| SEQ ID NO. 14: | Synechocystis PCCG8O3 homogentisate phythyltransferase polypeptide |
| SEQ ID NO. 15: | artificial codon usage optimized cDNA encoding Streptornyces avermitilis |
| | hydroxyphenyl-pyruvate dioxygenase (HPPDop) |
| SEQ ID NO. 16: | Streptomyces avermitilis hydroxyphenyl-pyruvate dioxygenase polypeptide |
| SEQ ID NO. 17: | Arabidopsis thaliana maleyl-acetoacetate isomerase (MAAI) cDNA |
| SEQ ID NO. 18: | Arabidopsis thaliana maleyl-acetoacetate isomerase (MAAI) polypeptide |
| SEQ ID NO. 19: | Arabidopsis thaliana γ-tocopherol methyltransferase cDNA |
| SEQ ID NO. 20: | Arabidopsis thaliana γ-tocopherol methyltransferase polypeptide |
| SEQ ID NO. 21: | Synechocystis PCC6803 3-methyl-6-phytylhydroquinone methyltransferase cDNA |
| SEQ ID NO. 22: | Synechocystis PCC6803 3-methyl-6-phytylhydroquinone methyltransferase polypeptide |
| SEQ ID NO. 23: | Nicotiana tabacurn geranylgeranyl pyrophosphate oxidoreductase cDNA |
| SEQ ID NO. 24: | Nicotiana tabacurn geranylgeranyl pyrophosphate oxidoreductase polypeptide |
| SEQ ID NO. 25: | Primer (5′-HGD Brassica napus) |
| | 5′-GTCGACGGNCCNATNGGNGCNAANGG-3′ |
| SEQ ID NO. 26: | Primer (3′-NOS terminator) |
| | 5′-AAGCTTCCGATCTAGTAACATAGA-3′ |
| SEQ ID NO. 27: | Primer (5′-35S promoter) |
| | 5′-ATTCTAGACATGGAGTCAAAGATTCAAATAGA-3′ |
| SEQ ID NO. 28: | Primer (3′-OCS terminator) |
| | 5′-ATTCTAGAGGACAATCAGTAAATTGAACGGAG-3′ |
| SEQ ID NO. 29: | Primer (5′-MAAI A. thaliana) |
| | 5′-atgtcgacATGTCTTATGTTACCGAT-3′ |
| SEQ ID NO. 30: | Primer (3′-MAAI A. thaliana) |
| | 5′-atggatccCTGGTTCATATGATACA-3′ |
| SEQ ID NO. 31: | Primer (5′-FAAH A. thaliana) |
| | 5′-atgtcgacGGAAACTCTGAACCATAT-3′ |
| SEQ ID NO. 32: | Primer (3′-FAAH A. thaliana) |
| | 5′-atggtaccGAATGTGATGCCTAAGT-3′ |
| SEQ ID NO. 33: | Primer (3′-HGD Brassica napus) |
| | 5′-GGTACCTCRAACATRAANGCCATNGTNCC-3′ |
| SEQ ID NO. 34: | Primer (5′-legumin promoter) |
| | 5′-GAATTCGATCTGTCGTCTCAAACTC-3′ |
| SEQ ID NO. 35: | Primer (3′-legumin promoter) |
| | 5′-GGTACCGTGATAGTAAACAACTAATG-3′ |
| SEQ ID NO. 36: | Primer (5′-transit peptide) |
| | 5′-ATGGTACCTTTTTTGCATAAACTTATCTTCATAG-3′ |
| SEQ ID NO. 37: | Primer (3′-transit peptide) |
| | 5′-ATGTCGACCCGGGATCCAGGGCCCTGATGGGTCCCATTTTCCC-3′ |
| SEQ ID NO. 38: | Primer (5′-NOS terminator) |
| | 5′-GTCGACGAATTTCCCCGAATCGTTC-3′ |
| SEQ ID NO. 39: | Primer (3′-NOS terminator II) |
| | 5′-AAGCTTCCGATCTAGTAACATAGA-3′ |
| SEQ ID NO. 40: | Primer (5′-legumin promoter II) |
| | 5′-AAGCTTGATCTGTCGTCTCAAACTC-3′ |
| SEQ ID NO. 41: | Arabidopsis thaliana maleyl-acetoacetate isomerase (MAAI) gene (fragment) |
| SEQ ID NO. 42: | Arabidopsis thaliana fumaryl acetoacetate hydrolase (FAAH) gene (fragment) |
| SEQ ID NO. 43: | Primer (5′-35S promoter) |
| | 5′-ATGAATTCCATGGAGTCAAAGATTCAAATAGA-3′ |
| SEQ ID NO. 44: | Primer (3′-OCS terminator) |
| | 5′-ATGAATTCGGACAATCAGTAAATTGAACGGAG-3′ |

### EXAMPLES

The invention is illustrated in greater detail in the use examples which follow with reference to the appended figures. Abbreviations with the following meanings are used:

| | |
|---|---|
| A = 35S promoter | B = HGD in antisense orientation |
| C = OCS terminator | D = legumin B promoter |
| E = FNR transit peptide | F = HPPDop |
| | (HPPD with optimized codon usage) |
| G = NOS terminator | H = MAAI in antisense orientation |
| I = FAAH in antisense orientation | |

The direction of arrows in the figures indicates in each case the direction in which the genes in question are read. In the figures:

FIG. 1 shows a schematic representation of the vitamin E biosynthetic pathway in plants;

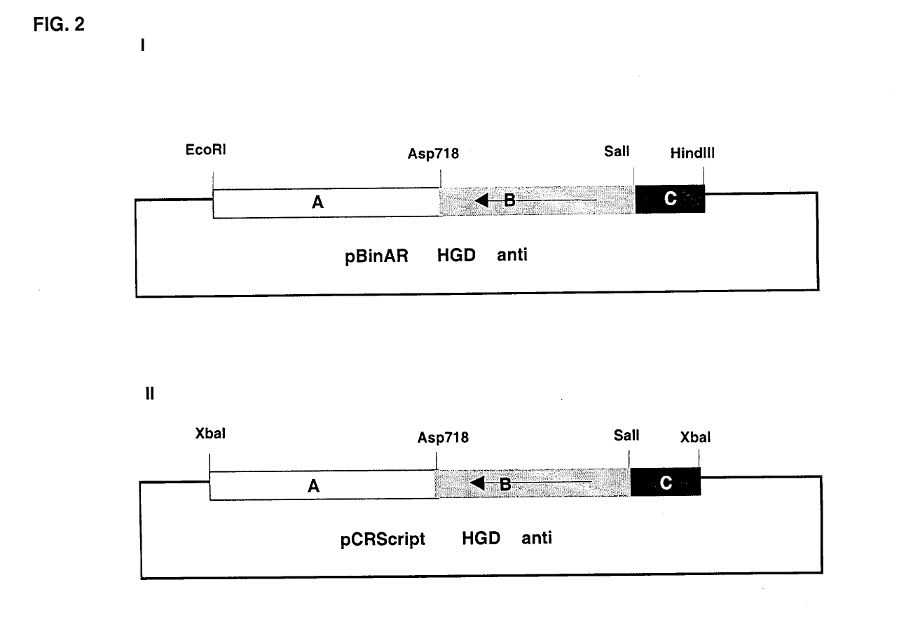

FIG. 2 shows construction schemes of the anti-HGD-coding plasmids pBinARHGDanti (I) and pCRScriptHGDanti (II);

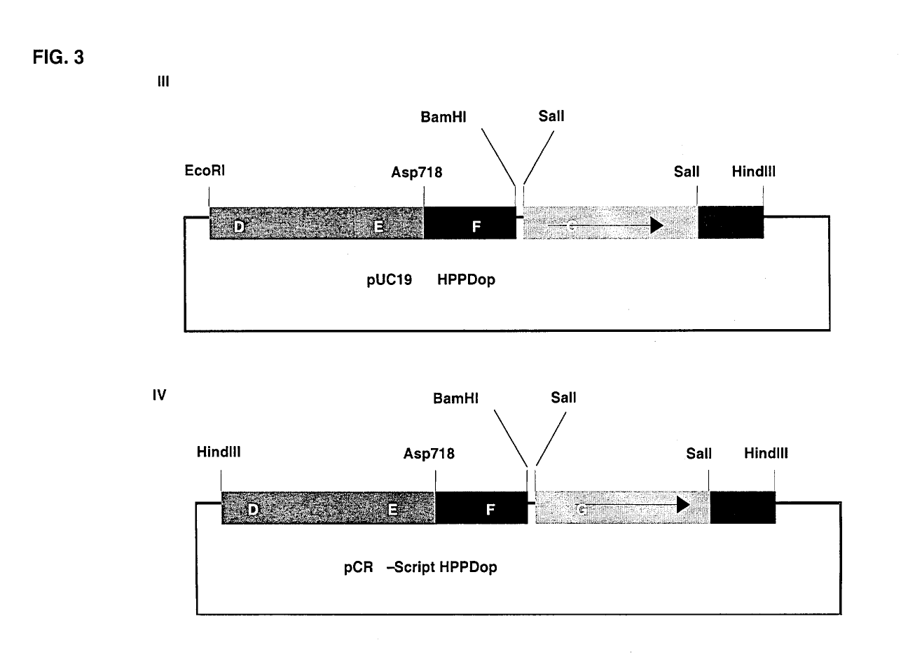

FIG. 3 shows construction schemes of the HPPDop-coding plasmids pUC19HPPDop (III) and pCRScriptHPPDop (IV);

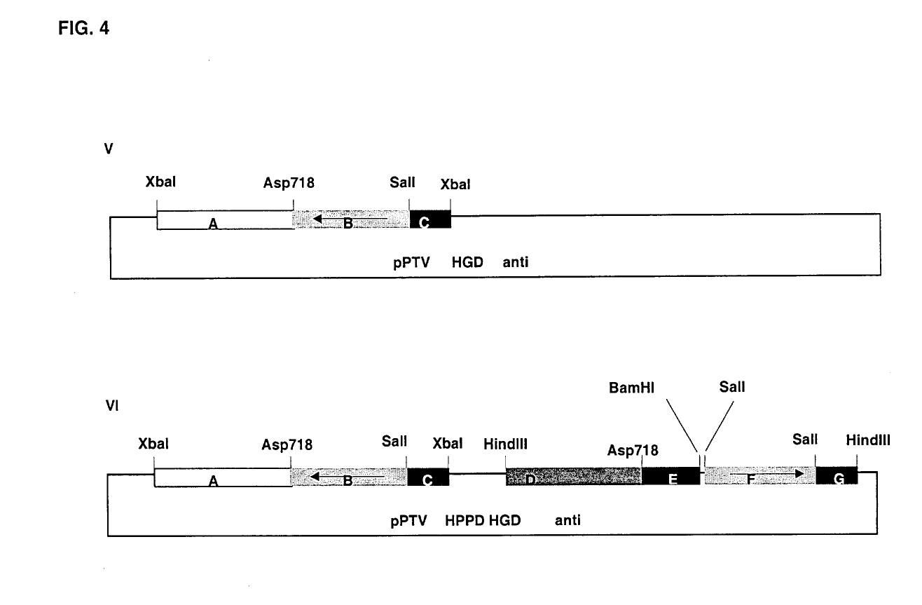

FIG. 4 shows construction schemes of the transformation vectors pPTVHGDanti (V) and of the bifunctional transformation vector pPTV HPPDop HGD anti (VI), which expresses HPPDop in the seeds of transformed plants while simultaneously suppressing the expression of the endogenous HGD;

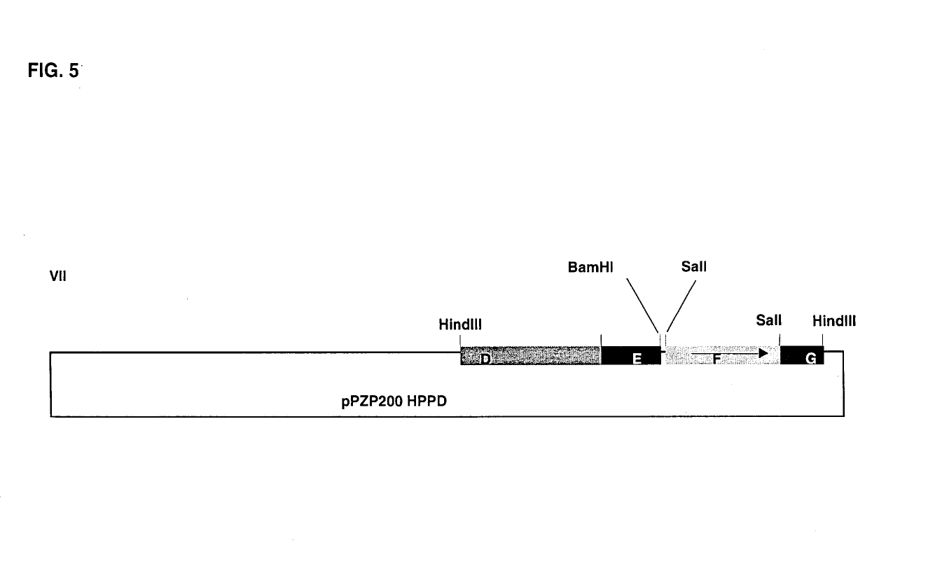

FIG. 5 shows a construction scheme of the transformation vector pPZP200HPPDop (VII).

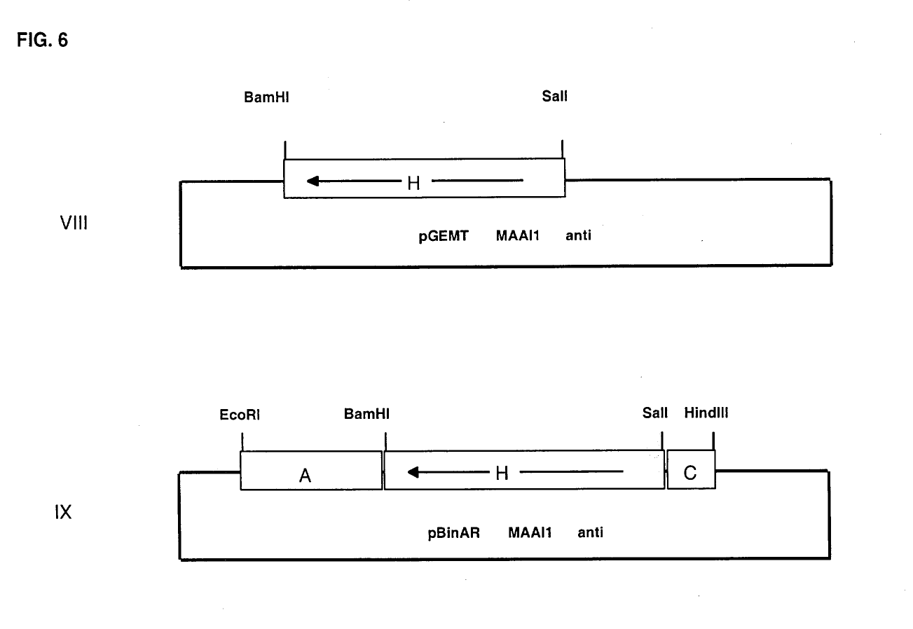

FIG. 6 shows construction schemes of the transformation vectors PGEMT MAAI1 anti (VIII) and pBinAR MAAI1 anti (IX);

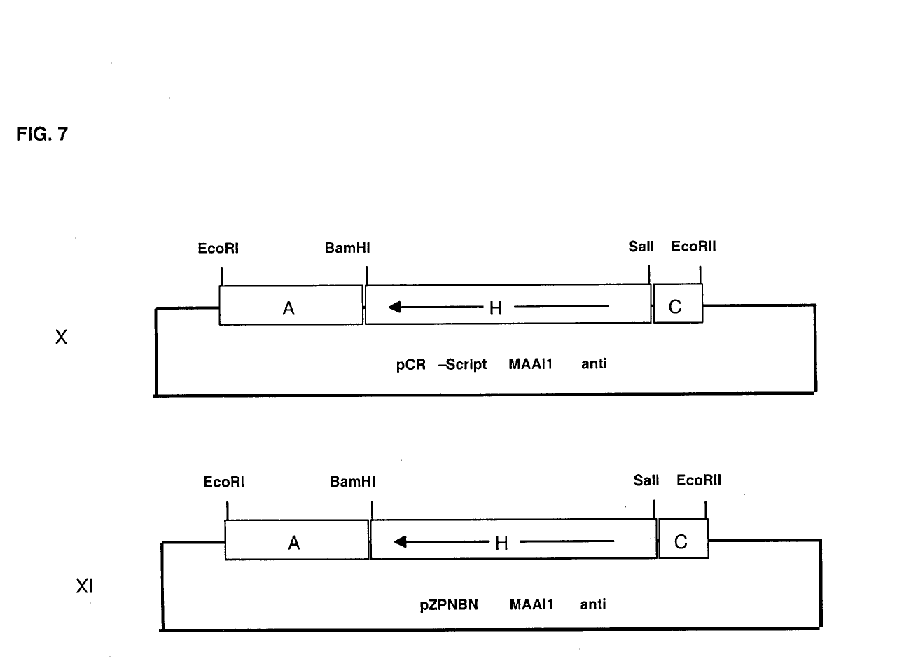

FIG. 7 shows construction schemes of the transformation vectors pCR-Script MAAI1 anti (X) and pZPNBN MAAI1 anti (XI);

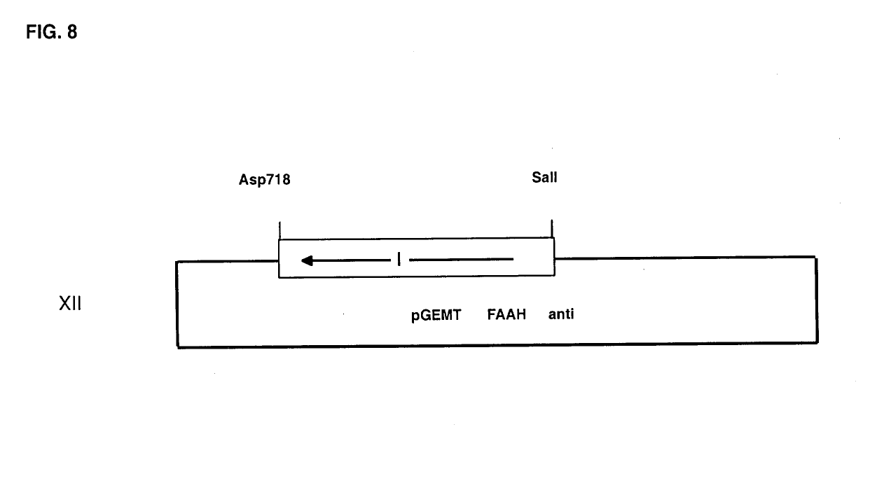

FIG. 8 shows the construction scheme of the transformation vector pGEMT FAAH anti (XII);

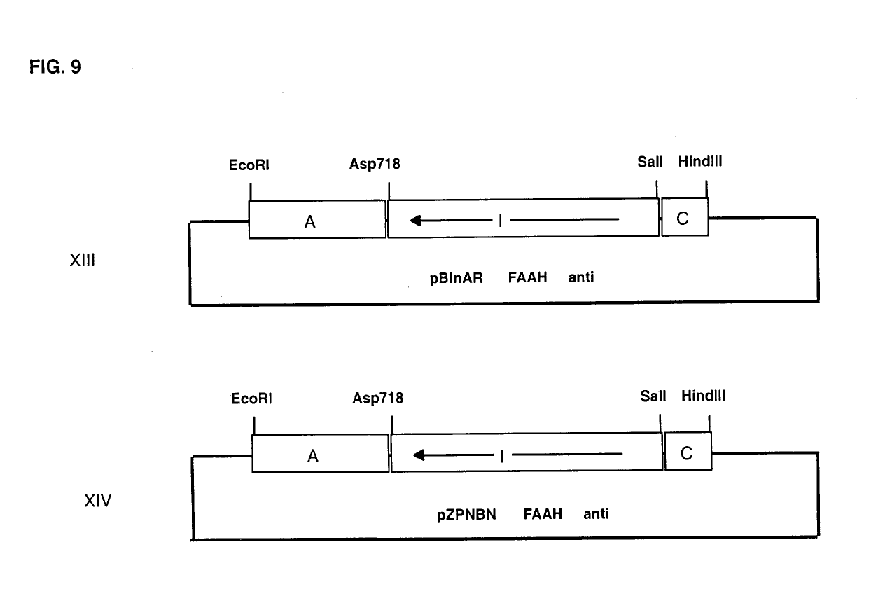

FIG. 9 shows construction schemes of the transformation vectors pBinAR FAAH anti (XIII) and pZPNBN FAAH anti (XIV).

### GENERAL METHODS

The chemical synthesis of oligonucleotides can be carried out for example in the known manner by the phosphoamidite method (Voet, Voet, 2nd Edition, Wiley Press New York, pp. 896-897). The cloning steps carried out within the present invention such as, for example, restriction cleavages, agarose gel electrophoresis, purification of the DNA fragments, transfer of nucleic acids to nitrocellulose and nylon membranes, linking DNA fragments, transformation of E. coli cells, bacterial cultures, phage replication and sequence analysis of recombinant DNA, were carried out as described by Sambrook et al. (1989) Cold Spring Harbor Laboratory Press; ISBN 0-87969-309-6. Recombinant DNA molecules were sequenced using a Licor laser fluorescence DNA sequencer (supplied by MWG Biotech, Ebersbach) using the method of Sanger (Sanger et al., Proc. Natl. Acad. Sci. USA 74 (1977), 5463-5467).

### EXAMPLE 1

### Cloning a Hydroxyphenyl-Pyruvate Dioxygenase (HPPD) with a DNA Sequence Optimized for Expression in Brassica napus

The amino acid sequence of the Streptomyces avermitilis hydroxyphenyl-pyruvate dioxygenase (HPPD) (Accession No. U11864, SEQ ID NO: 16) was backtranslated to a DNA sequence taking into consideration the codon usage in Brassica napus (oilseed rape). The codon usage was determined by means of the database http://www.dna.affrc.go.jp/˜nakamura/index.html. The derived sequence was synthesized by ligating overlapping oligonucleotides followed by PCR amplification, attaching SalI cleavage sites (Rouwendal, G J A; et al, (1997) PMB 33: 989-999) (SEQ ID NO: 15). The correctness of the sequence of the synthetic gene was verified by sequencing. The synthetic gene was cloned to the vector pBluescript II SK+ (Stratagene). (This codon-optimized sequence is subsequently also termed HPPDop.)

### EXAMPLE 2

### Cloning a Brassica napus Homogentisate Dioxygenase (HGD)

a) Isolating Total RNA from Brassica napus Flowers

Open flowers were harvested from Brassica napus var. Westar and frozen in liquid nitrogen. The material was subsequently reduced to a powder in a mortar and taken up in Z6 buffer (8 M guanidinium hydrochloride, 20 mM MES, 20 mM EDTA, brought to pH 7.0 with NaOH; immediately prior to use, 400 ml of mercaptoethanol/100 ml of buffer were added). The suspension was then transferred into reaction vessels and extracted by shaking with one volume of phenol/chloroform/isoamyl alcohol 25:24:1. After centrifugation for 10 minutes at 15,000 rpm, the supernatant was transferred into a new reaction vessel and the RNA was precipitated with 1/20 volume of 1N acetic acid and 0.7 volume of (absolute) ethanol. After a further centrifugation step, the pellet was first washed in 3M sodium acetate solution and, after another centrifugation step, in 70% strength ethanol. The pellet was subsequently dissolved in DEPC (diethylpyrocarbonate) water and the RNA concentration determined photometrically.

b) Preparation of cDNA from Total RNA from Brassica napus Flowers

20 mg of total RNA were first treated with 3.3 ml of 3M sodium acetate solution and 2 ml of 1M magnesium sulfate solution and the mixture was made up to an end volume of 10 ml with DEPC water. 1 ml of RNase-free DNase (Boehringer Mannheim) was added, and the mixture was incubated for 45 minutes at 37 degrees. After the enzyme had been removed by extracting by shaking with phenol/chloroform/isoamyl alcohol, the RNA was precipitated with ethanol and the pellet was taken up in 100 ml of DEPC water. 2.5 mg of RNA from this solution were transcribed into cDNA by means of a cDNA kit (Gibco BRL) following the manufacturer's instructions.

c) PCR Amplification of a Part-Fragment of the Brassica napus HGD

Oligonucleotides which had been provided with an SalI restriction cleavage site at the 5′ end and with an Asp718 restriction cleavage site at the 3′ end were derived for a PCR by aligning the DNA sequences of the known homogentisate dioxygenases (HGDs) from Arabidopsis thaliana (Accession No. U80668), Homo sapiens (Accession No. U63008) and Mus musculus (Accession No. U58988). The oligonucleotide at the 5′ end comprises the sequence:

5′-GTCGACGGNCCNATNGGNGCNAANGG-3′ (SEQ ID NO: 25),

starting with base 661 of the Arabidopsis gene. The oligonucleotide at the 3′ end comprises the sequence:

5′-GGTACCTCRAACATRAANGCCATNGTNCC-3′ (SEQ ID NO: 33),

starting with base 1223 of the Arabidopsis gene, N in each case denoting inosine and R denoting the incorporation of A or G into the oligonucleotide.

The PCR reaction was carried out with TAKARA Taq polymerase following the manufacturer's instructions. 0.3 mg of the cDNA was employed as template. The PCR program was:

1 cycle at: 94° C. (1 min)

5 cycles at: 94° C. (4 sec), 50° C. (30 sec), 72° C. (1 min)

5 cycles at: 94° C. (4 sec), 48° C. (30 sec), 72° C (1 min)

25 cycles at: 94° C. (4 sec), 46 degrees (30 sec), 72 degrees (1 min)

1 cycle at: 72 degrees (30 min)

The fragment was purified by NucleoSpin Extract (Macherey und Nagel) and cloned into vector PGEMT (Promega) following the manufacturer's instructions. The correctness of the fragment was verified by sequencing.

### EXAMPLE 3

### Generation of a Plant Transformation Construct for Overexpressing the HPPD with Optimized DNA Sequence (HPPDop) and Eliminating HGD

To generate plants which express HPPDop in seeds and in which the expression of the endogenous HGD is suppressed by antisense technology, a binary vector which contains both gene sequences was constructed (FIG. 4, construct VI).

a) Generation of an HPPDop Nucleic Acid Construct

To this end, the components of the cassette for expressing HPPDop, composed of the legumin B promoter (Accession No. X03677), the spinach ferredoxin:NADP+ oxidoreductase transit peptide (FNR; Jansen, T, et al (1988) Current Genetics 13, 517-522) and the NOS terminator (present in pBI101 Accession No. U12668) were first provided with the necessary restriction cleavage sites using PCR.

The legumin promoter was amplified from plasmid plePOCS (Bäumlein, H, et al. (1986) Plant J. 24, 233-239) with the upstream oligonucleotide:

5′-GAATTCGATCTGTCGTCTCAAACTC-3′ (SEQ ID NO: 34)

and the downstream oligonucleotide:

5′-GGTACCGTGATAGTAAACAACTAATG-3′ (SEQ ID NO: 35)

by means of PCR and cloned into vector PCR-Script (Stratagene) following the manufacturer's instructions.

The transit peptide was amplified with plasmid pSK-FNR (Andrea Babette Regierer “Molekulargenetische Ansätze zur Veränderung der Phosphat-Nutzungseffizienz von höheren Pflanzen” [Molecular genetic approaches for modifying the phosphate utilization efficiency of higher plants], P+H Wissenschaftlicher Verlag, Berlin 1998 ISBN: 3-9805474-9-3) by means of PCR using the 5′ oligonucleotide:

5′-ATGGTACCTTTTTTGCATAAACTTATCTTCATAG-3′ (SEQ ID NO: 36)

and the 3′ oligonucleotide:

5′-ATGTCGACCCGGGATCCAGGGCCCTGATGGGTCCCATTTTCCC-3′ (SEQ ID NO: 37)

The NOS terminator was amplified from plasmid pBI101 (Jefferson, R. A., et al (1987) EMBO J. 6 (13), 3901-3907) by means of PCR using the 5′ oligonucleotide:

5′-GTCGACGAATTTCCCCGAATCGTTC-3′ (SEQ ID NO: 38)

and the 3′ oligonucleotide

5′-AAGCTTCCGATCTAGTAACATAGA-3′ (SEQ ID NO: 26)

The amplicon was cloned in each case into vector pCR-Script (Stratagene) following the manufacturer's instructions.

For the nucleic acid constructs, the NOS terminator was first recloned as SalI/HindIII fragment into a suitably cut pUC19 vector (Yanisch-Perron, C., et al (1985) Gene 33, 103-119). The transit peptide was subsequently introduced into this plasmid as Asp718/SalI fragment. The legumin promoter was then cloned in as EcoRI/Asp718 fragment. The gene HPPDop was introduced into this construct as SalI fragment (FIG. 3, construct III).

The finished cassette in pUC19 was used as template for a PCR, using the oligonucleotide:

5′-AAGCTTGATCTGTCGTCTCAAACTC-3′ (SEQ ID NO: 40)

for the legumin promoter and the oligonucleotide:

5′-AAGCTTCCGATCTAGTAACATAGA-3′ (SEQ ID NO: 39)

for the NOS terminator. The amplicon was cloned into pCR-Script and termed pCR-ScriptHPPDop (FIG. 3, construct IV).

d) Generation of an AntiHGD Nucleic Acid Construct

To switch off HGD by means of antisense technology, the gene fragment was cloned as SalI/Asp718 fragment into vector pBinAR (Höfgen, R. und Willmitzer, L., (1990) Plant Sci. 66: 221-230), in which the 35S promoter and the OCS terminator are present (FIG. 2, construct I). The construct acted as template for a PCR reaction with the oligonucleotide:

5′-ATTCTAGACATGGAGTCAAAGATTCAAATAGA-3′ (SEQ ID NO: 27),

which is specific for the 35S promoter sequence; and the oligonucleotide:

5′-ATTCTAGAGGACAATCAGTAAATTGAACGGAG-3′ (SEQ ID NO: 28),

which is specific for the OCS terminator sequence.

The amplicon was cloned into vector pCR-Script (Stratagene) and termed pCRScriptHGDanti (FIG. 2, construct II).

c) Preparation of the Binary Vector

To construct a binary vector for transforming oilseed rape, the construct HGDanti from pCRScriptHGDanti was first cloned as XbaI fragment into vector pPTV (Becker, D., (1992) PMB 20, 1195-1197) (FIG. 4, construct V). The construct LegHPPDop from pCRScriptHPPDop was inserted into this plasmid as HindIII fragment. This plasmid was termed pPTVHPPDopHGDanti (FIG. 4, construct VI).

### EXAMPLE 4

### Generation of Constructs for the Cotransformation for Overexpressing HPPDop and Switching off HGD in Brassica napus Plants

To cotransform plants with HPPDop and antiHGD, the construct legumin B promoter/transit peptide/HPPDop/NOS was excised from vector pCRScriptHPPDop (FIG. 3, construct IV) as HindIII fragment and inserted into the correspondingly cut vector pPZP200 (Hajdukiewicz, P., et al., (1994) PMB 25(6): 989-94) (FIG. 5, construct VII). This plasmid was used later for cotransforming plants together with vector pPTVHGDanti (FIG. 4, construct V) of Example 3 c).

### EXAMPLE 5

### Cloning a Genomic Fragment of the Arabidopsis thaliana Maleyl-Acetoacetate Isomerase

a) Isolation of Genomic DNA from A. thaliana leaves:

The extraction buffer used has the following composition:

1 volume of DNA extraction buffer (0.35M sorbitol, 0.1 M Tris, 5 mM EDTA, pH 8.25 HCl)

1 volume of nuclei lysis buffer (0.2M Tris-HCl pH 8.0, 50 mM EDTA, 2 M NaCl, 2% hexadecyltrimethylammonium bromide (CTAB))

0.4 volume of 5% sodium sarcosyl

0.38 g/100 ml sodium bisulfite

100 mg of leaf material of A thaliana were harvested and frozen in liquid nitrogen. The material was subsequently reduced to a powder in a mortar and taken up in 750 μl of extraction buffer. The mixture was heated for 20 minutes at 65° C. and subsequently extracted by shaking with one volume of chloroform/isoamyl alcohol (24:1). After centrifugation for 10 minutes at 10,000 rpm in a Heraeus pico-fuge, the supernatant was treated with one volume of isopropanol, and the DNA thus precipitated was again pelleted for 5 minutes at 10,000 rpm. The pellet was washed in 70% strength ethanol, dried for 10 minutes at room temperature and subsequently dissolved in 100 μl of TE RNase buffer (10 mM Tris HCl pH 8.0, 1 mM EDTA pH 8.0, 100 mg/l RNase).

b) Cloning the Gene for the Arabidopsis thaliana MAAI

Using the protein sequence of mouse (Mus musculus) MAAI, the A. thaliana MAAI gene was identified by means of BLAST search in the NCBI database (http://www.ncbi.nlm.nih.gov/BLAST/) (Genbank Acc.-No. AAC78520.1). The sequence is annotated in Genbank as putative glutathione S-transferase. The corresponding DNA sequence was determined by means of the ID numbers of the protein sequence, and oligonucleotides were derived. An SalI restriction cleavage site was added to the 5′ end of each of the oligonucleotides and a BamHI restriction cleavage site to the 3′ end of each of the nucleotides. The oligonucleotide at the 5′ end encompasses the sequence

5′-atgtcgacATGTCTTATGTTACCGAT-3′ (SEQ ID NO: 29)

starting with base 37 of the cDNA, the first codon, the oligonucleotide at the 3′ end comprises the sequence

5′-atggatccCTGGTTCATATGATACA-3′ (SEQ ID NO: 30)

starting with base pair 803 of the cDNA sequence. The PCR reaction was carried out using Taq polymerase (manufacturer: TaKaRa Shuzo Co., Ltd.). The composition of the mix was as follows: 10 μl buffer (20 mM Tris-HCl pH 8.0, 100 mM KCl, 0,1 mM EDTA, 1 mM DTT, 0.5% Tween20, 0.5% Nonidet P-40, 50% glycerol), in each case 100 pmol of the two oligonucleotides, in each case 20 nM of dATP, dCTP, dGTP, dTTP, 2.5 units Taq polymerase, 1 μg of genomic DNA, distilled water to 100 μl. The PCR program was:

5 cycles at: 94° C. (4 sec), 52° C. (30 sec), 72° C. (1 min)

5 cycles at: 94° C. (4 sec), 50° C. (30 sec), 72° C. (1 min)

25 cycles at: 94° C. (4 sec), 48° C. (30 sec), 72° C. (1 min)

The amplified fragment (SEQ ID NO: 41) was purified by means of Nucleo-Spin Extract (Macherey-Nagel) and cloned into the Promega vector pGEMTeasy following the manufacturer's instructions (FIG. 6, construct VIII). The correctness of the fragment was verified by sequencing. By means of the restriction cleavage sites which had been added to the sequence by the primers, the gene was cloned into the correspondingly cut vector pBinAR (Höfgen, R. und Willmitzer, L., (1990) Plant Sci. 66: 221-230) as SalI/BamHI fragment (FIG. 6, construct IX). This vector contains the cauliflower mosaic virus 35S promoter and the OCS termination sequence. The construct acted as template for a PCR reaction with the oligonucleotide

5′-ATGAATTCCATGGAGTCAAAGATTCAAATAGA-3′ (SEQ ID NO: 43),

which is specific for the 35S promoter sequence and the oligonucleotide

5′-ATGAATTCGGACAATCAGTAAATTGAACGGAG-3′ (SEQ ID NO: 44),

which is specific for the OCS terminator. An- EcoRI recognition sequence was added to both oligonucleotides. The PCR was carried out using Pfu polymerase (manufacturer: Stratagene). The composition of the mix was as follows: 10 μl of buffer (200 mM Tris HCl pH 8.8, 20 mM MgSO₄, 100 mM KCl, 100 mM ammonium sulfate, 1% Triton X-100, 1 g/l nuclease-free BSA), in each case 100 pmol of the two oligonucleotides, in each case 20 nM of DATP, dCTP, dGTP, dTTP, 2.5 units Pfu polymerase, 1 ng of plasmid DNA, distilled water to 100 μl. The PCR program was:

5 cycles at: 94° C. (4 sec), 52° C. (30 sec), 72° C. (2 min)

5 cycles at: 94° C. (4 sec), 50° C. (30 sec), 72° C. (2 min)

25 cycles at: 94° C. (4 sec), 48° C. (30 sec), 72° C. (2 min)

The PCR fragment was purified by means of Nucleo-Spin Extract (Macherey-Nagel) and cloned into vector pCR-Script (Stratagene) (FIG. 7, construct X).

### EXAMPLE 6

### Generation of the Binary Vector

To construct a binary vector for transforming Arabidopsis and oilseed rape, the construct from vector pCR-Script was cloned into vector pZPNBN as EcoRI fragment. pZPNBN is a pPZP200 derivative (Hajdukiewicz, P., et al., (1994) PMB 25(6): 989-94), into which a phosphinothricin resistance under the control of the NOS promoter had been inserted before the NOS terminator. (FIG. 7, construct XI)

### EXAMPLE 7

### Cloning a Genomic Fragment of the Arabidopsis thaliana Fumaryl-Acetoacetate Isomerase

A BLAST search was carried out by means of the protein sequence of the Emericella nidulans FAAH, and a protein sequence was identified from A. thaliana which had 59% homology. A. thaliana FAAH has the Accession number AC002131. The DNA sequence was determined by means of the ID number of the protein sequence, and oligonucleotides were derived.

An SalI restriction cleavage site was added to the 5′ oligonucleotide and an Asp718 restriction cleavage site was added to the 3′ oligonucleotide. The oligonucleotide at the 5′ end of FAAH comprises the sequence

5′-atgtcgacGGAAACTCTGAACCATAT-3′ (SEQ ID NO: 31)

starting with base 40258 of BAC F12F1, the oligonucleotide at the 3′ end comprises the sequence:

5′-atggtaccGAATGTGATGCCTAAGT-3′ (SEQ ID NO: 32)

starting with base pair 39653 of the BAC. The PCR reaction was carried out with Taq polymerase (manufacturer: TaKaRa Shuzo Co., Ltd.). The composition of the mix was as follows: 10 μl buffer (20 mM Tris-HCl pH 8.0, 100 mM KCl, 0.1 mM EDTA, 1 mM DTT, 0.5% Tween20, 0.5% Nonidet P-40, 50% glycerol), in each case 100 pmol of the two oligonucleotides, in each case 20 nM of dATP, dCTP, dGTP, dTTP, 2.5 units Taq polymerase, 1 μg genomic DNA, distilled water to 100 μl. The PCR program was:

5 cycles at: 94° C. (4 sec), 52° C. (30 sec), 72° C. (1 min)

5 cycles at: 94° C. (4 sec), 50° C. (30 sec), 72° C. (1 min)

25 cycles at: 94° C. (4 sec), 48° C. (30 sec), 72° C. (1 min)

The fragement (SEQ ID NO: 42) was purified by means of Nucleo-Spin Extract (Macherey-Nagel) and cloned into the Promega vector pGEMTeasy following the manufacturer's instructions (FIG. 8, construct XII).

The correctness of the fragment was verified by sequencing. By means of the restriction cleavage sites added to the sequence of the primers, the gene was cloned as SalI/Asp718 fragment into the correspondingly cut vector pBinAR (Höfgen, R. und Willmitzer, L., Plant Sci. 66: 221-230, 1990). This vector contains the cauliflower mosaic virus 35S promoter and the OCS termination sequence (FIG. 9, construct XIII).

To construct a binary vector for transforming Arabidopsis and oilseed rape, the construct from vector pBinAr was cloned into vector pZPNBN as EcoRI/HindIII fragment. pZPNBN is a pPZP200 derivative (Hajdukiewicz, P., et al., (1994) Plant Molecular Biology 25(6): 989-94), into which a phosphinothricin resistance under the control of the NOS promoter had been inserted before the NOS terminator. (FIG. 9, construct XIV).

### EXAMPLE 8

### Generation of Transgenic Arabidopsis thaliana Plants

Wild-type Arabidopsis thaliana plants (cv. Columbia) were transformed with Agrobacterium tumefaciens strain (EHA105) on the basis of a modification of Clough's and Bent's vacuum infiltration method (Clough, S. and Bent A., Plant J. 16(6):735-43, 1998) and Bechtold, et al. (Bechtold, N., et al., CRAcad Sci Paris. 1144(2):204-212, 1993). The Agrobacterium tumefaciens cells used had previously been transformed with plasmids pZPNBN-MAAIanti or pZPNBN-FAAHanti.

Seeds of the primary transformants were screened on the basis of their phosphinothricin resistance by planting seed by hand and spraying the seedlings with the herbicide Basta (phosphinothricin). Basta-resistant seedlings were singled out and used for biochemical analysis as fully-developed plants.

### EXAMPLE 9

### Generation of Transgeniic Oilseed Rape (Brassica napus) Plants

The generation of transgenic oilseed rape plants followed in principle the procedure of Bade, J. B. and Damm, B. (Bade, J. B. and Damm, B. (1995) in: Gene Transfer to Plants, Potrykus, I. and Spangenberg, G., eds, Springer Lab Manual, Springer Verlag, 1995, 30-38), which also indicates the composition of the media and buffers used.

The transformation was carried out with the Agrobacterium tumefaciens strain EHA105. Either plasmid pPTVHPPDopHGDanti (FIG. 4, construct VI) or cultures of agrobacteria with plasmids pPTVHGDanti (FIG. 4, construct V) and pPZP200HPPDop (FIG. 5, construct VII) which were mixed after culturing were used for the transformation. Seeds of Brassica napus var. Westar were surface-sterilized with 70% strength ethanol (v/v), washed for 10 minutes with water at 55° C., incubated for 20 minutes in 1% strength hypochlorite solution (25% v/v Teepol, 0.1% v/v Tween 20) and washed six times with sterile water for in each case 20 minutes. The seeds were dried for three days on filter paper and 10-15 seeds were germinated in a glass flask containing 15 ml of termination medium. Roots and apices were removed from several seedlings (approx. size 10 cm), and the hypocotyls which remained were cut into sections approx. 6 mm long. The approx. 600 explants thus obtained were washed for 30 minutes with 50 ml of basal medium and transferred into a 300 ml flask. After addition of 100 ml of callus induction medium, the cultures were incubated for 24 hours at 100 rpm.

Overnight cultures of the Agrobacterium strains were set up in Luria broth supplemented with kanamycin (20 mg/l) at 29° C., and 2 ml of this were incubated in 50 ml of Luria broth medium without kanamycin for 4 hours at 29° C. until an OD₆₀₀ of 0.4-0.5 was reached. After the culture had been pelleted for 25 minutes at 2000 rpm, the cell pellet was resuspended in 25 ml of basal medium. The bacterial concentration of the solution was brought to an OD₆₀₀ of 0.3 by adding more basal medium. For the cotransformation, the solution of the two strains was mixed in equal parts.

The callus induction medium was removed from the oilseed rape explants using sterile pipettes, 50 ml of Agrobacterium solution were added, and the reaction wass mixed carefully and incubated for 20 minutes. The agrobacterial suspension was removed, the oilseed rape explants were washed for 1 minute with 50 ml of callus induction medium, and 100 ml of callus induction medium were subsequently added. Coculturing was carried out for 24 hours on an orbital shaker at 100 rpm. Coculturing was stopped by removing the callus induction medium and the explants were washed twice for in each case 1 minute with 25 ml and twice for 60 minutes with in each case 100 ml of wash medium at 100 rpm. The wash medium together with the explants was transferred into 15 cm Petri dishes, and the medium was removed using sterile pipettes.

For regeneration, in each case 20-30 explants were transferred into 90 mm Petri dishes containing 25 ml of shoot induction medium supplement with phosphinothricin. The Petri dishes were sealed with 2 layers of Leukopor and incubated at 25° C. and 2000 lux at photoperiods of 16 hours light/8 hours darkness. Every 12 days, the calli which developed were transferred to fresh Petri dishes containing shoot induction medium. All further steps for the regeneration of intact plants were carried out as described by Bade, J. B and Damm, B. (in: Gene Transfer to Plants, Potrykus, I. and Spangenberg, G., eds, Springer Lab Manual, Springer Verlag, 1995, 30-38).

### EXAMPLE 10

### Analysis of the Transgenic Plants

To verify that inhibition of HGD, MAAI and/or FAAH affects vitamin E biosynthesis in the transgenic plants, the tocopherol and tocotrienol contents in leaves and seeds of the plants (Arabidopsis thaliana, Brassica napus) which had been transformed with the above-described constructs were analyzed. To this end, the transgenic plants are grown in the greenhouse, and plants which express the antisense RNA of HGD, MAAI and/or FAAH are analyzed by means of a Northern blot analysis. The tocopherol content and the tocotrienol content in the leaves and seeds of these plants is determined. The plant material was disrupted by three indubations for 15 minutes in the Eppendorf shaker at 30° C., 1000 rpm in 100% methanol, and the supernatants obtained in each case were combined. Further incubation steps revealed no further liberation of tocopherols or tocotrienols. To avoid oxidation, the extracts obtained were analyzed directly after extraction with the aid of a Waters Allience 2690 HPLC system. Tocopherols and tocotrienols were separated using a reversed-phase column (ProntoSil 200-3-C30, Bischoff) using a mobile phase of 100% methanol and identified with reference to standards (Merck). The detection system used was the fluorescence of the substances (excitation 295 nm, emission 320 nm), which was detected with the aid of Jasco fluorescence detectors FP 920.

In all cases, the tocopherol and/or tocotrienol concentration in transgenic plants which additionally express a nucleic acid according to the invention is increased in comparison with untransformed plants.

We claim:

1. A process for the formation of vitamin E by influencing vitamin E biosynthesis, which comprises reducing homogentisate degradation by reducing homogentisate 1,2-dioxygenase (HGD) activity, maleyl-acetocacetate isomerase (MAAI) activity and/or fumaryl acetoacetate hydrolase (FAAH) activity.

2. A process as claimed in claim 1, wherein the MAAI activity and/or the FAAH activity is/are reduced and, simultaneously,
a) the conversion of homogentisate into vitamin E is improved or
b) the biosynthesis of homogentisate is improved.

3. A process as claimed in claim 1, wherein the HGD activity is reduced and, simultaneously,
a) the conversion of homogentisate into vitamin E is improved or
b) the TyrA gene is overexpressed.

4. A process for the increased formation of vitamin E by influencing vitamin E biosynthesis, which comprises
a) improving the conversion of homogentisate into vitamin E and simultaneously
b) improving the biosynthesis of homogentisate.

5. A process as claimed in any of claims 1 to 3, wherein the culture of a plant organism is treated with MAAI, HGD or FAAH inhibitors.

6. A nucleic acid construct comprising a nucleic acid sequence (anti-MAAI/FAAH) which is capable of reducing the MAAI activity or the FAAH activity, or one of its functional equivalents.

7. A nucleic acid construct as claimed in claim 6, additionally comprising
a) a nucleic acid sequence (pro-HG) which is capable of increasing homogentisate (HG) biosynthesis, or one of its functional equivalents; or
b) a nucleic acid sequence (pro-vitamin E) which is capable of increasing vitamin E biosynthesis starting from homogentisate, or one of its functional equivalents; or
c) a combination of a) and b).

8. A nucleic acid construct comprising a nucleic acid sequence (anti-HGD) which is capable of inhibiting HGD, or one of its functional equivalents.

9. A nucleic acid construct as claimed in claim 8 additionally comprising
a) a nucleic acid sequence encoding bifunctional chorismate mutase/prephenate dehydrogenase enzymes (TyrA) or one of its functional equivalents; or
b) a nucleic acid sequence (pro-vitamin E) which is capable of increasing vitamin E biosynthesis starting from homogentisate, or one of its functional equivalents; or
c) a combination of a) and b).

10. A nucleic acid construct comprising a nucleic acid sequence (pro-HG) which is capable of increasing homogentisate (HG) biosynthesis, or one of its functional equivalents, and simultaneously a nucleic acid sequence (pro-vitamin E), which is capable of increasing vitamin E biosynthesis starting from homogentisate, or one of its functional equivalents.

11. A nucleic acid construct as claimed in any of claims 6 to 10 comprising an anti-MAAI/FAAH sequence or anti-HGD sequence which
a) can be transcribed into an antisense nucleic acid sequence which is capable of inhibiting the MAAI/FAAH activity or the HGD activity, or
b) causes inactivation of MAAI/FAAH or HGD by homologous recombination, or
c) encodes a binding factor which binds to the MAAI/FAAH or HGD genes, thus reducing transcription of these genes.

12. A nucleic acid construct as claimed in either of claims 7 and 10 comprising a proHG sequence selected from among the genes encoding an HPPD, TyrA.

13. A nucleic acid construct as claimed in any of claims 7, 9 and 10 comprising a provitamin E sequence selected from among the genes encoding an HPGT, geranylgeranyl oxidoreduktase, 2-methyl-6-phytylplastoquinol methyltransferase, γ-tocopherol methyltransferase.

14. A recombinant vector comprising
a) a nucleic acid construct as claimed in any of claims 6 to 13; or
b) a nucleic acid encoding an HGD, MAAH or FAAH, and its functional equivalents, or
c) a combination of options a) and b).

15. A recombinant vector as claimed in claim 14, wherein the nucleic acid or nucleic acid constructs are linked functionally to a genetic control sequence and which is capable of transcribing sense or antisense RNA.

16. A transgenic organism transformed with a nucleic acid construct as claimed in any of claims 6 to 13 or a recombinant vector as claimed in claim 14 or 15.

17. A transgenic organism as claimed in claim 16 selected from among bacteria, yeasts, fungi, mosses, animal and plant organisms.

18. A cell culture, part, transgenic propagation material or fruit derived from a transgenic organism as claimed in claim 16 or 17.

19. The use of a transgenic organism as claimed in either of claims 16 or 17 or cell cultures, parts, transgenic propagation material or fruits derived therefrom as claimed in claim 18 as foodstuff or feedstuff or for isolating vitamin E.

20. An antibody, a protein-binding or a DNA-binding factor against polypeptides with HGD, MAAI or FAAH activity, their genes or cDNAs.

21. The use of polypeptides with HGD, MAAI or FAAH activity, their genes or cDNAs for finding HGD, MAAI or FAAH inhibitors.

22. A method of finding MAAI, HGD or FAAH inhibitors, which comprises measuring the enzymatic activity of MAAI, HGD or FAAH in the presence of a chemical compound where upon reduction of the enzymatic activity in comparison with the uninhibited activity the chemical compound constitutes an inhibitor.

23. The use of HGD, MAAI or FAAH inhibitors obtainable in accordance with a method as claimed in claim 22 as growth regulators.
